SEQUENCE LISTING (13 sequences)

SEQ ID NO: 1 - protein, 269 aa, Bordetella pertussis
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 2 - protein, 1464 aa, Bordetella pertussis
MDKDESALRQLVDMALVGYDGVVEELLALPSEESGDLAGGRAKREKAEFALFSEAPNGDEPIGQDARTWFYFPKYRPVAVSNLKKMQVAIRARLEPESLILQWLIALDVYLGVLIAALSRTVISDLVFEYVKARYEIYYLLNRVPHPLATAYLKRRRQRPVDRSGRLGSVFEHPLWFAYDELAGTVDLDADIYEQALAESIERRMDGEPDDGSLDTAEHDVWRLCRDGINRGEQAIFQASGPYGVVADAGYMRTVADLAYADALADCLHAQLRIRAQGSVDSPGDEMPRKLDAWEIAKFHLAATQQARVDLLEAAFALDYAALRDVRVYGDYRNALALRFIKREALRLLGARRGNASTMPAVAAGEYDEIVASGAANDAAYVSMAAALIAGVLCDLESAQRTLPVVLARFRPLGVLARFRRLEQETAGMLLGDQEPEPRGFISFTDFRDSDAFASYAEYAAQFNDYIDQYSILEAQRLARILALGSRMTVDQWCLPLQKVRHYKVLTSQPGLIARGIENHNRGIEYCLGRPPLTDLPGLFTMFQLHDSSWLLVSNINGELWSDVLANAEVMQNPTLAALAEPQGRFRTGRRTGGWFLGGPATEGPSLRDNYLLKLRQSNPGLDVKKCWYFGYRQEYRLPAGALGVPLFAVSVALRHSLDDLAAHAKSALYKPSEWQKFAFWIVPFYREIFFSTQDRSYRVDVGSIVFDSISLLASVFSIGGKLGSFTRTQYGNLRNFVVRQRIAGLSGQRLWRSVLKELPALIGASGLRLSRSLLVDLYEIFEPVPIRRLVAGFVSATTVGGRNQAFLRQAFSAASSSAGRTGGQLASEWRMAGVDATGLVESTSGGRFEGIYTRGLGPLSECTEHFIVESGNAYRVIWDAYTHGWRVVNGRLPPRLTYTVPVRLNGQGHWETHLDVPGRGGAPEIFGRIRTRNLVALAAEQAAPMRRLLNQARRVALRHIDTCRSRLALPRAESDMDAAIRIFFGEPDAGLRQRIGRRLQEVRAYIGDLSPVNDVLYRAGYDLDDVATLFNAVDRNTSLGRQARMELYLDAIVDLHARLGYENARFVDLMAFHLLSLGHAATASEVVEAVSPRLLGNVFDISNVAQLERGIGNPASTGLFVMLGAYSESSPAIFQSFVNDIFPAWRQASGGGPLVWNFGPAAISPTRLDYANTDIGLLNHGDISPLRARPPLGGRRDIDLPPGLDISFVRYDRPVRMSAPRALDASVFRPVDGPVHGYIQSWTGAEIEYAYGAPAAAREVMLTDNVRIISIENGDEGAIGVRVRLDTVPVATPLILTGGSLSGCTTMVGVKEGYLAFYHTGKSTELGDWATAREGVQALYQAHLAMGYAPISIPAPMRNDDLVSIAATYDRAVIAYLGKDVPGGGSTRITRHDEGAGSVVSFDYNAAVQASAVPRLGQVYVLISNDGQGARAVLLAEDLAWAGSGSALDVLNERLVTLFPAPV

SEQ ID NO: 3 - protein, 403 aa, Bordetella pertussis
MAPLLVLGFASGLPLALSSGTLQAWATVENVSLQSIGFLTLAGTAYTLKFLWAPLIDRYVPPFLGRRRGWMLLTQVLLAAAIMVMGMLSPGSALLPLALVAVLVAFLSASQDIAFDAYSTDVLRQEERGAGAAMRVMGYRLAMIVSGGLALIVADRWLGWGNTYVLMGGLMLACALGTLWAPEPERPANPPRDLGAAVVEPFREFFSRRGAIDMLLLIVLYKLGDAFAGALSTTFLLRGAGFSATEVGTVNKVLGLAATIVGALAGGSIMTRWGLYRSLMAFGLLQAVSNLGYWLIAVSPKNLYLMGLAVGVENLCGGLGTASFVALLMAMCRQQFSATQFALLSALAAVGRTYLAGPLTPVLVEWLDWPGFFIVTVLIALPGLWLLRLRRNVIDELDAQTAR

SEQ ID NO: 4 - protein, 491 aa, Escherichia coli
MSSQYLRIFQQPRSAILLILGFASGLPLALTSGTLQAWMTVENIDLKTIGFFSLVGQAYVFKFLWSPLMDRYTPPFFGRRRGWLLATQILLLVAIAAMGFLEPGTQLRWMAALAVVIAFCSASQDIVFDAWKTDVLPAEERGAGAAISVLGYRLGMLVSGGLALWLADKWLGWQGMYWLMAALLIPCIIATLLAPEPTDTIPVPKTLEQAVVAPLRDFFGRNNAWLILLLIVLYKLGDAFAMSLTTTFLIRGVGFDAGEVGVVNKTLGLLATIVGALYGGILMQRLSLFRALLIFGILQGASNAGYWLLSITDKHLYSMGAAVFFENLCGGMGTSAFVALLMTLCNKSFSATQFALLSALSAVGRVYVGPVAGWFVEAHGWSTFYLFSVAAAVPGLILLLVCRQTLEYTRVNDNFISRTEYPAGYAFAMWTLAAGISLLAVWLLLLTMDALDLTHFSFLPALLEVGVLVALSGVVLGGLLDYLALRKTHLM

SEQ ID NO: 5 - DNA, 32 bp, synthetic construct
  misc_feature 1..32: note = Synthetic Oligonucleotide A
  source 1..32: mol_type = other DNA
tataaatcgatattcctgctggtttcgttctc

SEQ ID NO: 6 - DNA, 30 bp, synthetic construct
  misc_feature 1..30: note = Synthetic Oligonucleotide B
  source 1..30: mol_type = other DNA
tatagctagcaagttgggaaacgacaccac

SEQ ID NO: 7 - DNA, 25 bp, synthetic construct
  misc_feature 1..25: note = Synthetic Oligonucleotide C
  source 1..25: mol_type = other DNA
taagaagcaaaataagccaggcatt

SEQ ID NO: 8 - DNA, 30 bp, synthetic construct
  misc_feature 1..30: note = Synthetic Oligonucleotide D
  source 1..30: mol_type = other DNA
tataccatggcgccgctgctggtgctgggc

SEQ ID NO: 9 - DNA, 30 bp, synthetic construct
  misc_feature 1..30: note = Synthetic Oligonucleotide E
  source 1..30: mol_type = other DNA
tatatctagacgctggccgtaaccttagca

SEQ ID NO: 10 - DNA, 30 bp, synthetic construct
  misc_feature 1..30: note = Synthetic Oligonucleotide F
  source 1..30: mol_type = other DNA
tatagaattcgctcggttcgctggtcaagg

SEQ ID NO: 11 - DNA, 30 bp, synthetic construct
  misc_feature 1..30: note = Synthetic Oligonucleotide G
  source 1..30: mol_type = other DNA
tatatctagagcaatgccgattcatcttta

SEQ ID NO: 12 - DNA, 30 bp, synthetic construct
  misc_feature 1..30: note = Synthetic Oligonucleotide H
  source 1..30: mol_type = other DNA
tatatctagagcggcctttattgcttttcc

SEQ ID NO: 13 - DNA, 30 bp, synthetic construct
  misc_feature 1..30: note = Synthetic Oligonucleotide I
  source 1..30: mol_type = other DNA
tataaagcttctcatgcacgccggcttctc

(19) United States
(12) Patent Application Publication
(10) Pub. No.: US 2025/0032600 A1
(43) Pub. Date: Jan. 30, 2025

(54) Attenuated Bordetella Strains

(71) Applicants: Institut Pasteur de Lille, Lille Cedex (FR); Institut National de la Santé et de la Recherche Médicale (INSERM), Paris Cedex 18 (FR)

(72) Inventors: Camille Locht, Brussels (BE); Nathalie Mielcarek, Blandain (FR); Anne Sophie Debrie, La Madeleine (FR); Dominique Raze, Gruson (FR); Julie Bertout, Pont-à-Marcq (FR)

(21) Appl. No.: 18/807,921

(22) Filed: Aug. 17, 2024

Related U.S. Application Data
(63) Continuation of application No. 17/650,487, filed on Feb. 9, 2022.
Application No. 17/650,487 is a continuation of application No. 16/787,767, filed on Feb. 11, 2020, now Pat. No. 11,285,201.
Application No. 16/787,767 is a continuation of application No. 16/278,562, filed on Feb. 18, 2019, now Pat. No. 10,610,580.
Application No. 16/278,562 is a division of application No. 15/626,331, filed on Jun. 19, 2017, now Pat. No. 10,258,681.
Application No. 15/626,331 is a continuation of application No. 14/860,210, filed on Sep. 21, 2015, now Pat. No. 9,730,995.
Application No. 14/860,210 is a continuation of application No. 14/658,817, filed on Mar. 16, 2015, now Pat. No. 9,180,178.
Application No. 14/658,817 is a division of application No. 12/224,895, filed on Nov. 19, 2008, now Pat. No. 9,119,804.
(60) Provisional application No. 60/817,430, filed on Jun. 30, 2006.
(60) Provisional application No. 60/780,827, filed on Mar. 10, 2006.

(51) Int. Cl. A61K 39/02; A61K 39/00; A61P 31/04; C07K 14/235; C12R 1/01

(52) U.S. Cl. CPC A61K 39/099; A61P 31/04; C07K 14/235; A61K 2039/522; A61K 2039/523; A61K 2039/543; A61K 2039/55; A61K 2039/575; C12R 2001/01

(57) ABSTRACT

A mutated Bordetella strain comprising at least a mutated ptx gene, a deleted or mutated dnt gene and a heterologous ampG gene is provided. The attenuated mutated Bordetella strain can be used in an immunogenic composition or a vaccine for the treatment or prevention of a Bordetella infection. Use of the attenuated Bordetella strain for the manufacture of a vaccine or immunogenic composition, as well as methods for protecting mammals against infection by Bordetella are also provided.

### CROSS-REFERENCE TO RELATED APPLICATIONS

The present application is a continuation application of U.S. patent application Ser. No. 17/650,487 filed on Feb. 9, 2022 which is a continuation application of U.S. patent application Ser. No. 16/787,767 filed on Feb. 11, 2020 (now U.S. Pat. No. 11,285,201), which is a continuation application of U.S. patent application Ser. No. 16/278,562 filed on Feb. 18, 2019 (now U.S. Pat. No. 10,610,580), which is a division of U.S. patent application Ser. No. 15/626,331 filed on Jun. 19, 2017 (now U.S. Pat. No. 10,258,681), which is a continuation application of U.S. patent application Ser. No. 14/860,210 filed on Sep. 21, 2015 (now U.S. Pat. No. 9,730,995), which is a continuation application of U.S. patent application Ser. No. 14/658,817 filed on Mar. 16, 2015 (now U.S. Pat. No. 9,180,178), which is a divisional application of U.S. nonprovisional patent application Ser. No. 12/224,895 filed on Nov. 19, 2008 (now U.S. Pat. No. 9,119,804) as a national stage entry application under 35 U.S.C. 371 of international patent application number PCT/EP2007/01942, filed on Mar. 7, 2007, which designated the U.S. and claims the priority of U.S. provisional patent application Ser. No. 60/817,430 filed on Jun. 30, 2006 and U.S. provisional patent application Ser. No. 60/780,827 filed on Mar. 10, 2006, which are incorporated herein by reference in their entirety.

### SEQUENCE LISTING

The instant application contains a Sequence Listing which has been submitted electronically in XML format, created on Aug. 5, 2024, is named 7056-0185 SL.xml, and is 18,396 bytes in size. The Sequence Listing is hereby incorporated by reference in its entirety.

### FIELD OF THE INVENTION

The present invention relates to a mutated Bordetella strain comprising at least a mutated ptx gene, a deleted or mutated-dnt gene and a heterologous ampG gene. The attenuated mutated Bordetella strain can be used in an immunogenic composition or a vaccine for the treatment or prevention of a Bordetella infection. Use of the attenuated Bordetella strain for the manufacture of a vaccine or immunogenic compositions, as well as methods for protecting mammals against infection by Bordetella also form a part of the invention.

### BACKGROUND OF THE INVENTION AND RELATED PRIOR ART

Pertussis is still among the principal causes of death world-wide, and its incidence is increasing even in countries with high vaccine coverage. Although all age groups are susceptible, it is most severe in infants too young to be protected by currently available vaccines.

Whooping cough or pertussis is a severe childhood disease responsible for high mortality rates before the introduction of effective vaccines in the second half of the 20th century. The success of these vaccines has led to the opinion that the disease is essentially under control, although world-wide 200,000 to 400,000 pertussis-linked deaths are still recorded annually, and the disease still ranks sixth among the causes of mortality due to infectious agents [1]. Although mostly prevalent in developing countries, the disease is also re-emerging in the developed world [2, 3], including the U.S.A., where the incidence has increased five-fold over the last twenty years [4]. Unexpectedly, the epidemiology of pertussis has changed in countries with high vaccine coverage, where cases of adolescent and adult pertussis are increasingly frequent [5]. This is probably due to progressive waning of vaccine-mediated immunity during adolescence. Often atypical and therefore difficult to diagnose, pertussis is generally not life-threatening in adults and in many cases remains unnoticed. However, infected adults constitute an important reservoir for transmission of the disease to very young children, too young to be fully vaccinated, and therefore at risk to develop severe disease associated with high mortality rates.

Pertussis vaccination usually begins at two months of age, and full protection requires at least three immunizations at one- to two-month intervals. Therefore, infants are not fully protected before the age of 6 months using the currently available vaccines. To reduce the incidence of pertussis in the very young and most vulnerable age groups, early immunization, possibly at birth, would thus be highly desirable. However, numerous studies in humans and in animal models have suggested that the neonatal immune system is too immature to effectively induce vaccine-mediated protective immunity [6, 7]. Especially the IFN-y production, indicative of a Th1 response that is essential to the development of protective immunity to pertussis [8], appears to be significantly reduced in human newborns, compared to older children or adults [9]. This is also reflected by the fact that significant amounts of antigen-specific IFN-y are only produced after several months (≥6 months) in children vaccinated with pertussis vaccines, especially with acellular vaccines (aPV) [10].

Natural infection with Bordetella pertussis has long been considered to induce strong and long-lasting immunity, that wanes much later than vaccine-induced immunity [5, 11]. Furthermore, infection with B. pertussis induces measurable antigen-specific Th1 type immune responses even in very young children (as young as one month of age) [12]. These observations suggest that live vaccines applicable by the nasal route in order to mimic as closely as possible natural infection, may be attractive alternatives over the currently available vaccines.

There are many vaccinating compositions to treat Bordetella infections known in the art. However, these immunogenic compositions are not used to treat newborn children or in cases where an epidemic and rapid protective immunity is required.

Thus, French Patent FR 0206666 discloses live Bordetella strains that have been rendered deficient in at least two toxins chosen from PTX, DNT, AC and TCT. This patent discloses the over expression of an endogenous ampG gene by the addition of a strong promoter, and the addition of 11 terminal amino acids of the ampG gene from E. coli.

Mielcarek et al, Vaccine (2006; 2452:52154-52-55) disclose a strain of Bordetella pertussis attenuated of PTK, DTN- and TCr for use in the immunization of mice. This reference discloses that to reduce the production of tracheal cytotoxin, the ampG gene should be overexpressed. However, upon further evaluation, the authors realized that by over-expressing the ampG gene, there is an increase in tracheal cytotoxin and not a decrease as was originally thought.

Mielcarek et al in Advance Drug Delivery Review 51 (2001) pgs. 55-69 disclose that live vaccines can induce systemic and mucosal responses when administered by the oral or nasal route.

Roduit et al in Infection and Immunity (2002 July; 70 (7): 3521-8} describe vaccinating neonatals and infants with mutated Bordetella strains with a DTP composition.

Mattoo et al, in Frontiers of Bioscience 6, e168-e186 (2001), suggest replacing the endogenous ampG gene in Bordetella with the E. coli ampG gene, which resulted in a decrease in the amount of TCT produced.

Thus, the prior art although disclosing various types of vaccinating compositions fails to address the problem of providing a vaccine or immunogenic composition that can provide protection to a newborn prior to six months. Furthermore, the prior art fails to disclose an immunogenic or a vaccine that provides rapid protective immunity against a Bordetella infection. The prior art also fails to disclose an immunogenic composition or vaccine that provides a rapid protective immunity against a Bordetella infection, said protective immunity increasing over at least the next two months following vaccination.

Therefore, it is an object of the present invention to overcome the deficiencies in the prior art.

It is another object of the present invention to produce a live attenuated vaccine candidate or immunogenic composition through genetic attenuation of a Bordetella strain such as B. pertussis or B. parapertussis to diminish pathogenicity, while maintaining the ability to colonize and induce protective immunity.

It is another object of the present invention to produce a vaccine or immunogenic composition that induces protection in newborns after a single intranasal administration that is superior to the protection provided by the current aPV.

It is yet another object of the present invention to provide protection against infection with Bordetella parapertussis, as well as Bordetella pertussis which was not seen after vaccination with aPV.

Another object of the present invention is to induce strong protective immunity in newborns against Bordetella infection.

Yet another object of the present invention is to provide a vaccine or immunogenic composition that induces mucosal and systemic immunity.

It is another object of the present invention to produce a live attenuated Bordetella pertussis strain to be given as a single-dose nasal vaccine in early life, called BPZE1.

It is yet another object of the present invention to provide a vaccine that can not only be used to vaccinate newborns, but can be used in all mammals of any age in the case of an epidemic of whooping cough.

Another object of the present invention is to provide a vaccine against Bordetella infection that induces a rapid protective immunity and/or a protective immunity that increases over at least the next two months after the vaccination.

Yet another object of the present invention is to provide prevention or treatment against Bordetella infection that is relatively low in production costs.

These and other objects are achieved by the present invention as evidenced by the summary of the invention, description of the preferred embodiments and the claims.

### SUMMARY OF THE INVENTION

The present invention provides a mutated Bordetella strain comprising at least a mutated pertussis toxin (ptx) gene, a deleted or mutated dermonecrotic toxin (dnt) gene, and a heterologous ampG gene.

In another aspect the present invention relates to an immunogenic composition comprising a mutated Bordetella strain comprising at least a mutated pertussis toxin (ptx) gene, a deleted or mutated pertussis dermonecrotic toxin (dnt) gene, and a heterologous ampG gene.

In yet another aspect the present invention provides a vaccine comprising the attenuated Bordetella strain comprising at least a mutated pertussis toxin (ptx) gene, a deleted or mutated pertussis dermonecrotic toxin (dnt) gene, and a heterologous ampG gene.

In still another aspect, the present invention provides the use of an attenuated Bordetella strain comprising at least a mutated ptx gene, a deleted or mutated dnt gene, and a heterologous ampG gene for the manufacture of a vaccine for the prevention of a Bordetella infection.

In yet another aspect, the present invention provides the use of an attenuated Bordetella strain comprising at least a mutated ptx gene, a deleted or mutated dnt gene, and a heterologous ampG gene for the manufacture of a vaccine for the induction of an immune response directed preferentially toward the Th1 pathway against said attenuated Bordetella.

Also provided is a method of protecting a mammal against disease caused by infection by Bordetella pertussis and Bordetella parapertussis comprising administering to said mammal in need of such treatment a mutated Bordetella strain comprising at least a mutated ptx gene, a deleted or mutated dnt gene, and a heterologous ampG gene.

A method of providing a rapid protective immunity against a Bordetella infection comprising administering to said mammal in need of such treatment a mutated Bordetella strain comprising at least a mutated ptx gene, a deleted or mutated dnt gene, and a heterologous ampG gene is also part of the present invention.

A method of providing a rapid protective immunity against a Bordetella infection comprising administering to a mammal in need of such treatment a mutated Bordetella strain comprising at least a mutated ptx gene, a deleted or mutated dnt gene, and a heterologous ampG gene or a vaccine comprising said mutated Bordetella strain, wherein said method provides further an increase in said protective immunity over at least two months after vaccination is still another aspect of the present invention.

Use of the mutated Bordetella strain comprising at least a mutated ptx gene, a deleted or mutated dnt gene and a heterologous ampG gene for the preparation of a multivalent vaccine (i.e., a vaccine for preventing or treating infections caused by different pathogens) to treat respiratory diseases is yet another aspect of the present invention.

Use of an attenuated Bordetella strain of the invention, by administration to mammals in need of a rapid protective immunity against a Bordetella infection, wherein said protective immunity increases over at least two months after administration, is also part of the present invention.

A method to provide a mucosal response and a systemic response to treat or protect against Bordetella infections in mammals is still another aspect of the present invention.

### BRIEF DESCRIPTION OF THE DRAWINGS

FIG. 1 is a bar graph illustrating the TCT present in culture supernatants of BPSM and BPZE1 expressed as means of nM/OD_{540 nm} (±standard error) of 3 separate cultures for each strain.

FIG. 2 is an immunoblot analysis of PTX production in the culture supernatants of BPSM (lane 1) and BPZE1 (lane 2). The sizes of the Mr markers are expressed in kDa and given in the left margin.

FIG. 3 is a Southern-blot analysis of the dnt locus in BPSM (lane 1) and BPZE (lane 2). The lengths of the size markers are indicated in base pairs (bp) are shown in the left margin.

FIG. 4 is a graph illustrating the growth rates of BPSM (black line) and BPZE1 (dotted line) in liquid culture.

FIG. 5 are electron micrographs representative of BPSM (left) and BPZE1 (right) grown in liquid medium for 24 h.

FIG. 6 is a graph illustrating the in vitro adherence of BPSM (black columns) and BPZE1 (white columns) to human pulmonary epithelial A549 cells (left) and murine macrophage-like J774 cells (right). The results are expressed as means of percentages of binding bacterial relative to the bacteria present in the inoculum from three different experiments.

FIG. 7 is a graph illustrating lung colonization by BPSM (black lines) and BPZE1 (dotted lines) of adult mice infected intranasally with 106 CFU of BPZE1 or BPSM. The results are expressed as mean (±standard error) CFUs from three to four mice per group and are representative of two separate experiments. P=0.004.

FIG. 8 are photographs of a histological analysis of lungs from BPZE1 (upper panel) or BPSM-infected (middle panel) adult mice compared to controls given PBS (lower panel). One week after infection, the lungs were aseptically removed and fixed in formaldehyde. Sections were stained with hematoxylin and eosin and examined by light microscopy.

FIG. 9 are graphs illustrating the protection against B. pertussis in (a) adult and (b) infant mice or B. parapertussis in infant mice (d). Mice immunized with BPZE1, aPV or PBS (naive) were challenged with BPSM (a and b) or B. parapertussis (d), and lung CFU counts were determined 3 h (white bars) or 7 days (black bars) later. Results are expressed as mean (±standard error) CFUs from 3-4 mice per group and are representative of two separate experiments. (b,*, P=0.009; d,*, P=0.007) (c) CFU counts 3 h after BPSM challenge in adult mice vaccinated with BPZE1 or aPV, compared to controls. Results obtained from 3 separate experiments are expressed as percentages of CFUs of each mouse relative of the average of CFUs in non-immunized group from the same experiment.

FIG. 10 are bar graphs illustrating the immune responses induced by BPZE1 or aPV immunization. (a) Anti-FHA lgG(H+l) titers and (b) lgG1/IgG2a ratios before (white bars) or 1 week after BPSM challenge (black bars) in BPZE1 or aPV immunized mice, compared to controls. (c) IFN-y to IL-5 ratios produced by FHA-, PTX- or ConA-stimulated splenocytes from mice vaccinated 2 months before with BPZE1 (black bars) or aPV (white bars), compared to controls (gray bars). Antibodies and cytokines were measured in individual mice, and the results are expressed as mean values (±standard error) for 4 mice per group tested in triplicate.

FIG. 11 is the amino acid sequence of pertussis toxin (SEQ 10 N0:1) (islet-activating protein S1). The first 34 amino acids are the signal sequence, while amino acids 35 to 269 are the mature chain.

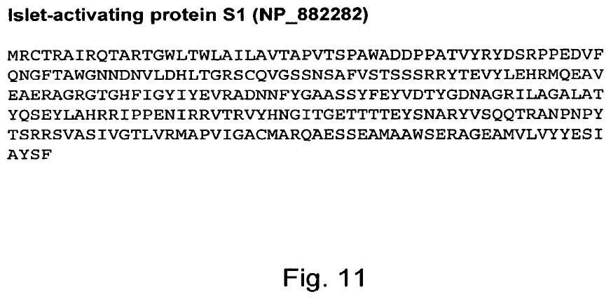

FIG. 12 is the amino acid sequence of dennonecrotic toxin (SEQ ID N0:2).

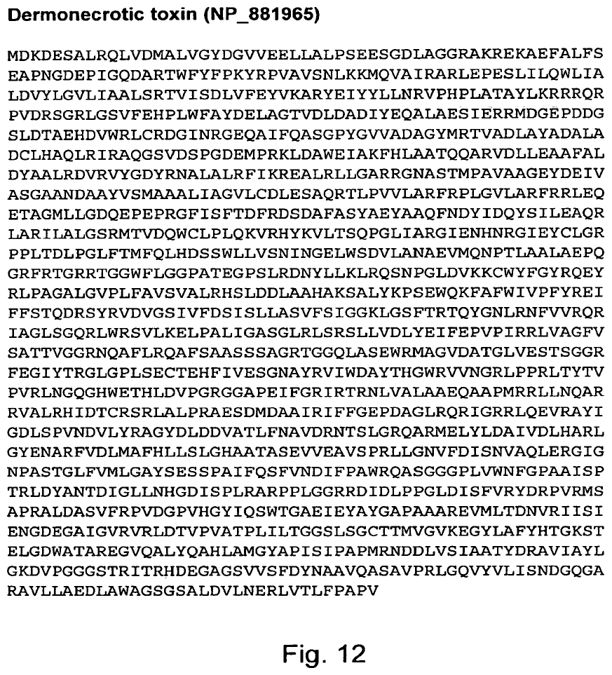

FIG. 13 is the amino acid sequence of .AmpG from Bordetella pertussis (SEQ ID N0:3).

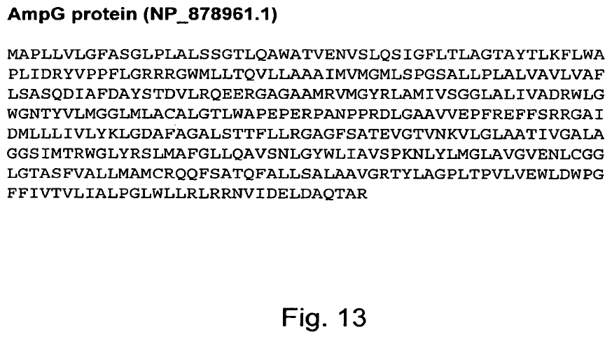

FIG. 14 is the amino acid sequence of AmpG from Escherichia coli (SEQ ID N0:4).

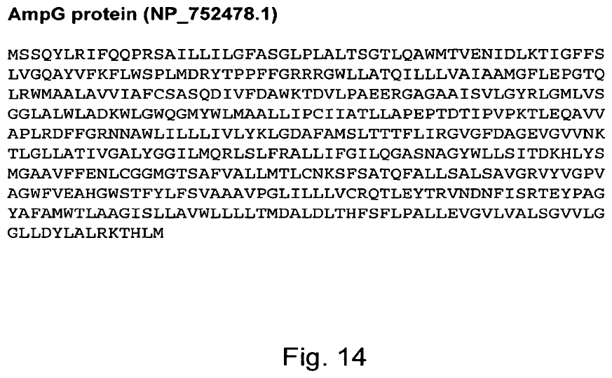

### DESCRIPTION OF THE PREFERRED EMBODIMENTS OF THE PRESENT INVENTION

As used herein, the abbreviation “PTX” refers to pertussis toxin, which synthesizes and secretes an ADP-ribosylating toxin. PTX is composed of six polypeptides S1 to S5, the enzymatically active moiety is called S1. PTX has a 34 amino acid signal sequence, while the mature chain consists of amino acids 35 to 269. PTX is the major virulence factor expressed by B. pertussis. The A moiety of these toxins exhibit ADP-ribosyltransferase activity and the B portion mediates binding of the toxin to host cell receptors and the translocation of A to its site of action (57).

As used herein the abbreviation “DNT” refers to pertussis dermonecrotic toxin, which is a heat labile toxin that induces localized lesions in mice and other laboratory animals when it is injected intradermally. It is lethal to mice when it is injected in low doses intravenously (58 to 61). DNT is considered to be a virulence factor for the production of turbinate atrophy in porcine atrophic rhinitis (62, 63).

As used herein the abbreviation “TCT” refers to tracheal cytotoxin, which is a virulence factor synthesized by Bordetellae. TCT is a peptidoglycan fragment and has the ability to induce interleukin-1 production and nitric oxide synthase. It has the ability to cause stasis of cilia and has lethal effects on respiratory epithelial cells.

The term “mammal” encompasses any of various warm-blooded vertebrate animals of the class Mammalia, including humans, characterized by a covering of hair on the skin and, in the female, milk-producing mammary glands for nourishing the young.

The term “attenuated” means a weakened, less virulent Bordetella strain that is capable of stimulating an immune response and creating protective immunity, but does not cause any illness.

The terminology “rapid protective immunity” means that immunity against Bordetella is conferred in a short time after administration of the mutated Bordetella strain of the present invention. By “short time” means vaccinated and challenged one week later. More specifically, there is a quick expansion of existing pathogen-specific peripheral lymphocytes, CDS+ cytotoxic effectors (CTLs) and CD4+ helper cells. The CD4+ helper cells induce B cell maturation and antibody production. Thus, lymphocytes with the memory pool are poised to rapidly proliferate at the time of subsequent infection.

The term “Bordetella strain” encompasses strains from Bordetella pertussis, Bordetella parapertussis and Bordetella bronchiseptica.

The expression “Bordetella infection” means an infection caused by at least one of the three following strains: Bordetella pertussis, Bordetella parapertussis and Bordetella bronchiseptica.

By “child” is meant a person or a mammal between 6 months and 12 years of age.

By the term “newborn” is meant a person or a mammal that is between 1 day old and 24 weeks of age.

The term “treatment” as used herein is not restricted to curing a disease and removing its causes but particularly covered means to cure, alleviate. Remove or lessen the symptoms associated with the disease of interest, or prevent or reduce the possibility of contracting any disorder or malfunction of the host body.

The terms “protection” and “prevention” are used herein interchangeably and mean that an infection by Bordetella is impeded.

“Prophylaxis vaccine” means that this vaccine prevents Bordetella infection upon future exposure.

By “preferentially towards the Th1 pathway” is meant that the Th1 pathway is favored over the Th2 pathway.

The term “immunogenic composition” means that the composition can induce an immune response and is therefore antigenic. By “immune response” means any reaction by the immune system. These reactions include the alteration in the activity of an organism immune system in response to an antigen and may involve, for example, antibody production, induction of cell-mediated immunity, complement activation or development of immunological tolerance

More specifically, the present invention provides at least a triple mutated Bordetella strain that can be used as an immunogenic composition or a vaccine. It will be appreciated that the at least triple mutated Bordetella strain contains a mutated ptx gene, a deleted or mutated dnt gene and a heterologous ampG gene. The heterologous ampG gene product reduces in large quantities the amount of tracheal cytotoxin that is produced.

The present invention is not limited to only the triple mutants described above. Other additional mutations can be undertaken such as adenylate cyclase (AC) deficient mutants (64), lipopolysaccharide (LPS) deficient mutants (65), filamentous hemagglutinin (FHA) (66) and any of the bvg-regulated components (67).

The starting strain which is mutated can be any Bordetella strain including Bordetella pertussis, Bordetella parapertussis and Bordetella bronchiseptica. In one aspect the starting strain used to obtain the mutated Bordetella strain is B. pertussis.

The construction of the mutated Bordetella strain starts with replacing the Bordetella ampG gene in the strain with a heterologous ampG gene. Any heterologous ampG gene can be used in the present invention. These include all those gram-negative bacteria that release very small amounts of peptidoglycan fragments into the medium per generation. Examples of gram-negative bacteria include, but are not limited to Escherichia coli, Salmonella, Enterobacteriaceae, Pseudomonas, Moraxella, Helicobacter, Stenotrophomonas, Legionella and the like.

By replacing the Bordetella ampG gene with a heterologous ampG gene, the amount of tracheal cytoxin (TCT) produced in the resulting strain expresses less than 1% residual TCT activity. In another embodiment, the amount of TCT toxin expressed by the resulting strain is between 0.6% to 1% residual TCT activity or 0.4% to 3% residual TCT activity or 0.3% to 5% residual TCT activity.

PTX is a major virulence factor responsible for the systemic effects of B. pertussis infections, as well as one of the major protective antigens. Due to its properties, the natural ptx gene is replaced by a mutated version so that the enzymatically active moiety S1 codes for an enzymatically inactive toxin, but the immunogenic properties of the pertussis toxin are not affected. This can be accomplished by replacing the arginine (Arg) at position 9 of the sequence with a lysine (Lys). Furthermore, a glutamic acid (Glu) at position 129 is replaced with a glycine (Gly).

Other mutations can also be made such as those described in U.S. Pat. No. 6,713,072, incorporated herein by reference, as well as any known or other mutations able to reduce the toxin activity to undetectable levels. Allelic exchange is first used to delete the ptx operon and then to insert the mutated version.

Finally, the dnt gene is then removed from the Bordetella strain by using allelic exchange. Besides the total removal, the enzymatic activity can also be inhibited by a point mutation. Since DNT is constituted by a receptor-binding domain in the N-terminal region and a catalytic domain in the C-terminal part, a point mutation in the dot gene to replace Cys-1305 to Ala-1305 inhibits the enzyme activity of DNT (68). DNT has been identified as an important toxin in Bordetella bronchiseptica and displays lethal activity upon injection of minute quantities (26).

Besides allelic exchange to insert the mutated ptx gene and the inhibited or deleted dot gene. the open reading frame of a gene can be interrupted by insertion of a genetic sequence or plasmid. This method is also contemplated in the present invention.

The triple mutated strain of the present invention is called a BPZE1 strain and has been deposited with the Collection Nationale de Cultures de Microorganismes (CNCM) in Paris, France on Mar. 9, 2006 under the number CNCM 1-3585. The mutations introduced into BPZE1 result in drastic attenuation, but allow the bacteria to colonize and persist. Thus, in another embodiment the present invention provides BPZE1, which can induce mucosal immunity and systemic immunity when administered. In another aspect the BPZE1 is administered intranasally.

The mutated Bordetella strains of the present invention can be used in immunogenic compositions. Such immunogenic compositions are useful to raise an immune response, either an antibody response and or preferably a T cell response in mammals. Advantageously, the T cell response is such that it protects a mammal against Bordetella infection or against its consequences.

The mutated Bordetella strains of the present invention can be used as live strains or chemically or heat-killed strains in the vaccines or immunogenic compositions. In one aspect, the live strains are used for nasal administration, while the chemically—or heat killed strains can be used for systemic or mucosal administration.

The immunogenic composition may further comprise a pharmaceutically suitable excipient or carrier and/or vehicle, when used for systemic or local administration. The pharmaceutically acceptable vehicles include, but are not limited to, phosphate buffered saline solutions, distilled water, emulsions such as an oil/water emulsions, various types of wetting agents sterile solutions and the like.

The immunogenic composition of the invention can also comprise adjuvants, i.e., any substance or compound capable of promoting or increasing a T-cell mediated response, and particularly a CD4⁺-mediated or CD8⁺-mediated immune response against the active principle of the invention. Adjuvants such as muramyl peptides such as MDP, IL-12, aluminium phosphate, aluminium hydroxide, Alum and/or Montanide® can be used in the immunogenic compositions of the present invention.

It would be appreciated by the one skilled in the art that adjuvants and emulsions in the immunogenic compositions are used when chemically or heat treated mutated Bordetella strains are used in the vaccines or immunogenic compositions.

The immunogenic compositions of the invention further comprise at least one molecule having a prophylactic effect against a Bordetella infection or the detrimental effects of Bordetella infection, such as a nucleic acid, a protein, a polypeptide, a vector or a drug.

The immunogenic composition of the invention is used to elicit a T-cell immune response in a host in which the composition is administered. All immunogenic compositions described above can be injected in a host via different routes: subcutaneous (s.c.), intradermal (i.d.), intramuscular (i.m.) or intravenous (i.v.) injection, oral administration and intranasal administration or inhalation.

When formulated for subcutaneous injection, the immunogenic composition or vaccine of the invention preferably comprises between 10 and 100 μg of the Bordetella strain per injection dose, more preferably from 20 to 60 μg/dose, especially around 50 μg/dose, in a sole injection

When formulated for intranasal administration, the Bordetella strain is administered at a dose of approximately 1×10³ to 1×10⁶ bacteria, depending on the weight and age of the mammal receiving it. In another aspect a dose of 1×10⁴ to 5×10⁶ can be used.

The mutated Bordetella strains of the present invention can be used as an attenuated vaccine to protect against future Bordetella infection. In this regard, an advantage of the present invention is that a single dose can be administered to mammals and the protection can last at least for a duration of longer than two months, particularly longer than six months. The vaccine of the present invention can be administered to newborns and protects against infection of whooping cough. This is especially crucial since the fatality rate from Bordetella pertussis infections is about 1.3% for infants younger than 1 month.

Moreover, the vaccines of the present invention can be used in adult mammals when there is an epidemic or in older adults over the age of 60, since their risk of complications maybe higher than that of older children or healthy adults.

The vaccines can be formulated with the physiological excipients set forth above in the same manner as in the immunogenic compositions. For instance, the pharmaceutically acceptable vehicles include, but are not limited to, phosphate buffered saline solutions, distilled water, emulsions such as an oil/water emulsions, various types of wetting agents sterile solutions and the like. Adjuvants such as muramyl peptides such as MDP. IL-12, aluminium phosphate, aluminium hydroxide, Alum and/or Montanide® can be used in the vaccines.

The vaccines of the present invention are able to induce high titers of serum IgG against FHA. The analysis of the antigen-specific cytokine patterns revealed that administration with the mutated attenuated Bordetella strains of the present invention favored a strong TH1 response.

The vaccines of the present invention provide high level of protection against a Bordetella infection i.e., a level of protection higher than 90%, particularly higher than 95%, more particularly higher than 99% (calculated 7 days after infection as detailed on example 9). The level of protection of the vaccine comprising the BPZE1 strain reaches more than 99.999% compared to non-vaccinated (naïve) mice, at least two months after vaccination.

The vaccines can be administered subcutaneous (s.c.), intradermal (i.d.), intramuscular (i.m.) or intravenous (i.v.) injection, oral administration and intranasal administration or inhalation. The administration of the vaccine is usually in a single dose. Alternatively, the administration of the vaccine of the invention is made a first time (initial vaccination), followed by at least one recall (subsequent administration), with the same strain, composition or vaccine, or with acellular vaccines, or a combination of both.

In one aspect, intranasal administration or inhalation of the vaccines is accomplished, which type of administration is low in costs and enables the colonization by the attenuated strains of the invention of the respiratory tract: the upper respiratory tract (nose and nasal passages, paranasal sinuses, and throat or pharynx) and/or the respiratory airways (voice box or larynx, trachea, bronchi, and bronchioles) and/or the lungs (respiratory bronchioles, alveolar duets, alveolar sacs, and alveoli)

Intranasal administration is accomplished with an immunogenic composition or a vaccine under the form of liquid solution, suspension, emulsion, liposome, a cream, a gel or similar such multiphasic composition. Solutions and suspensions are administered as drops. Solutions can also be administered as a fine mist from a nasal spray bottle or from a nasal inhaler. Gels are dispensed in small syringes containing the required dosage for one application.

Inhalation is accomplished with an immunogenic composition or a vaccine under the form of solutions, suspensions, and powders; these formulations are administered via an aerosol or a dry powder inhaler. Compounded powders are administered with insufflators or puffers.

Use of the mutated Bordetella strains comprising at least a mutated pix gene, a deleted or mutated dot gene and a heterologous ampG gene for the preparation of a multivalent vaccine to treat respiratory diseases is yet another aspect of the present invention. In this regard, the attenuated mutated Bordetella strain described above, can be used as a heterologous expression platform to carry heterologous antigens to the respiratory mucosa. Thus, such respiratory pathogens such as Neisseria, Pneumophila, Yersinia, Pseudomonas, Mycobacteria, Influenza and the like can prevent infection using the BPZE1 as a carrier.

Use of the live attenuated mutated Bordetella strains described herein for the manufacture of a vaccine for the treatment or prevention of Bordetella infection is also encompassed by the present invention. In this regard, the vaccine can be used for the simultaneous treatment or prevention of an infection by B. pertussis and B. parapertussis.

Use of the vaccine to provide rapid protective immunity in case of a pertussis epidemic is also encompassed by the present invention.

Use of the vaccine to provide a rapid protective immunity, increasing over the at least next two months following vaccination is also encompassed by the present invention.

The vaccine or immunogenic composition is also provided in a kit. The kit comprises the vaccine or immunogenic composition and an information leaflet providing instructions for immunization.

The present invention also relates to a method for inducing T-cell mediated immune response and particularly a CD4⁺-mediated immune response or a CD8⁺-mediated immune response, comprising administering the live attenuated Bordetella strains of the invention in a non-human mammal or a human mammal.

A method of protecting a mammal against disease caused by infection by Bordetella comprising administering to said mammal in need of such treatment a mutated Bordetella strain comprising at least a mutated ptx gene, a deleted or mutated dnt gene, and a heterologous ampG gene is another embodiment of the present invention. This method encompasses treating or preventing infections against Bordetella pertussis and/or Bordetella parapertussis. In one aspect the BPZE1 strain is used in this method.

Also a method of providing a rapid protective immunity against a Bordetella infection comprising administering to said mammal in need of such treatment a mutated Bordetella strain comprising at least a mutated ptx gene, a deleted or mutated dnt gene, and a heterologous ampG gene is encompassed by the present invention. In one aspect the BPZE1 strain is used in this method.

Moreover, the mutated live attenuated Bordetella strains of the present invention induce mucosal immunity, as well as systemic immunity. Thus, in another aspect the invention also relates to a method of inducing mucosal and systemic immunity by administering to a mammal in need of such treatment the mutated live attenuated Bordetella strains of the present invention. In one aspect the BPZE1 strain is used in this method.

Besides its role in the prevention and/or treatment of Bordetella infection, the mutated strain of the invention may be used as vector, to bear at least one further heterologous nucleic acid sequence encoding a RNA (such as antisense RNA) or a protein of interest. This means that the mutated strain bears at least one further heterologous nucleic acid sequence in addition to the heterologous ampG gene. In one aspect, the protein encoded by this at least one further heterologous nucleic acid sequence is a protein for which the expression is desired in the respiratory tract. In another aspect, the protein of interest is an antigen, such as a viral, a bacterial or a tumoral antigen, against which an immune response is desired. Therefore, the mutated Bordetella strain bearing at least one further heterologous nucleic acid sequence may also be used as a vaccine. The definitions given above for administration of the vaccine or immunogenic composition also apply to a vaccine comprising mutated Bordetella strain bearing at least one further heterologous nucleic acid sequence. Examples of heterologous proteins are antigens of pathogens causing infections of or diseases associated with the respiratory track: poliomyelitis, influenza (influenzavirus from Orthomyxoviridae family) or antigens from pneumococcus (such as Streptococcus pneumoniae).

A number of embodiments of the invention have been described. Nevertheless, it will be understood that various modifications may be made without departing from the spirit and scope of the invention.

### EXAMPLES

### Materials and Methods

### Example 1—Bordetella Strains and Growth Conditions

The B. pertussis strains used in this study were all derived from B. pertussis BPSM [13], and B. parapertussis is a streptomycin-resistant derivative of strain 12822 (kindly provided by Dr. N. Guiso, Institut Pasteur Paris, France). All Bordetella strains were grown on Bordet-Gengou (BG) agar (Difco, Detroit, Mich.) supplemented with 1% glycerol, 20% defibrinated sheep blood, and 100 μg/ml streptomycin. For cell adherence assays, exponentially growing B. pertussis was

inoculated at an optical density of 0.15 at 600 nm in 2.5 ml modified Stainer-Scholte medium [14] containing 1 g/l heptakis(2,6-di-o-methyl) β-cyclodextrin (Sigma) and supplemented with 65 μCi/ml L-[³⁵S]methionine plus L-[³⁵S]cysteine (NEN, Boston, Mass.) and grown for 24 h at 37° C. The bacteria were then harvested by centrifugation, washed three times in phosphate-buffered saline (PBS) and resuspended in RPMI 1640 (Gibco, Grand Island, N.Y.) at the desired density.

### Example 2-—Construction of B. pertussis BPZE1

To construct B. pertussis BPZE1, the B. pertussis ampG gene was replaced by Escherichia coli ampG using allelic exchange. A PCR fragment named met and located at position 49,149 to 49,990 of the B. pertussis genome, upstream of the B. pertussis ampG gene, was amplified using oligonucleotides A: 5′-TATAAATCGATATTCCTGCTGGTTTCGTTCTC-3′ (SEQ ID No:5) and B: 5′-TATAGCTAGCAAGTTGGGAAACGACACCAC-3′ (SEQ ID No:6), and B. pertussis BPSM [13] genomic DNA as a template. This 634 bp fragment was inserted into Topo PCRII (In Vitrogen Life Technology, Groningen. The Netherlands) and then excised as a ClaI-NheI fragment and inserted into ClaI- and NbeI-digested pBP23 [50], a suicide vector containing the E. coli ampG gene with flanking B. pertussis DNA of 618 bp (from position 50,474 to 51,092 of the B. pertussis genome) and 379 bp (from position 52,581 to 52,960 of the B. pertussis genome) at the 5′ and 3′ end of E. coli ampG, respectively. The resulting plasmid was transferred into E. coli SM10 [51], which was then conjugated with BPSM, and two successive homologous recombination events were selected as described [52]. Ten individual colonies were screened by PCR as follows. The colonies were suspended in 100 μl H₂O, heated for 20 min. at 95° C., and centrifuged for 5 min at 15,000×g. One μl of supernatants was then used as template for PCR using oligonucleotides A and C: 5′-TAAGAAGCAAAATAAGCCAGGCATT-3′ (SEQ ID No:7) to verify the presence of E. coli ampG and using oligonucleotides D: 5′-TATACCATGGCGCCGCTGCTGGTGCTGGGC-3′ (SEQ ID No:8) and E: 5′-TATATCTAGACGCTGGCCGTAACCTTAGCA-3′ (SEQ ID No:9) to verify the absence of B. pertussis ampG. One of the strains containing E. coli ampG and lacking B. pertussis ampG was then selected, and the entire ampG locus was sequenced. This strain was then used for further engineering.

The ptx genes were deleted from the chromosome of this strain as described [21] and then replaced by mutated ptx coding inactive PTX. The EcoRI fragment containing the mutated ptx locus from pPT-RE [16] was inserted into the EcoRI site of pJQ200mp18rpsl [53]. The resulting plasmid was integrated into the B. pertussis chromosome at the pix locus by homologous recombination after conjugation via E. coli SM10. The ptx locus in the chromosome of the resulting B. pertussis strain was sequenced to confirm the presence of the desired mutations. Toxin production was analyzed by immunoblotting using a mix of monoclonal antibodies IB7 [54] specific for subunit S1, and 11E6 [55] specific for subunits S2 and S3 of PTX.

Finally, the dnt gene was deleted from the resulting B. pertussis strain as the dnt flanking regions were amplified by PCR using BPSM genomic DNA as a template and oligonucleotides F: 5′-TATAGAATTCGCTCGGTTCGCTGGTCAAG G-3′ (SEQ ID No:10) and G: 5′-TATATCTAGAGCAATGCCGATTCATCTTTA-3′ (SEQ ID No:11) for the dnt upstream region, and H: 5′-TATATCTAGAGCGGCCTT TATTGCTTTTCC-3′ (SEQ ID No:12) and I: 5′-TATAAAGCTTCTCATGCACGCCG GCTTCTC-3′ (SEQ ID No:13) for the dnt downstream region, as primers. The resulting 799-bp and 712-bp DNA fragments were digested with EcoRI/XbaI and XbaI/HindIII, respectively, and linked together using the Fast Link kit (Epicentre Biotechnologies, Madison, Wis.). The ligated fragment was amplified by PCR using oligonucleotides F and I, and the 1505-bp PCR fragment was then inserted into pCR2.1-Topo (Invitrogen), re-isolated from the resulting plasmid as an EcoRI fragment and inserted into the unique EcoRI site of pJQmp200rpsL18. The resulting plasmid was introduced into B. pertussis by conjugation via E. coli SM10, Successful deletion of the dnt gene by allelic exchange was verified by Southern blot analysis on PvuII-digested B. pertussis genomic DNA using the PCR fragment corresponding to the dnt upstream region as a probe. The probe was labeled with digoxigenin (DIG) using the DIG Easy Hyb labeling kit (Roche, Meylan, France). The sizes of the hybridizing bands were determined from the migration distance of the Dig-labeled DNA molecular marker III (Roche). The dnt locus of this final strain, named BPZE1 was sequenced.

### Example 3—Analysis of TCT Production

For sensitive quantitation of TCT production, culture supernatants of B. pertussis grown to logarithmic phase were collected, subjected to solid phase extraction [15] and derivatized with phenylisothiocyanate (PITC, Pierce). The resulting phenylthiocarbamyl (PTC) derivatives were separated by reversed-phase HPLC using a C8 column (Perkin Elmer) and detected at 254 nm. The amount of B. pertussis PTC-TCT in each sample was determined by comparing the peak area and elution time with an identically processed TCT standard.

### Example 4—Cell-Adherence Assay

To analyze adherence properties of the B. pertussis strains, their attachment rates to the human pulmonary epithelial cell line A549 (ATCC no CCL-185) and the murine macrophage cell line J774 (ATCC no TIB-67) were measured as previously described [16].

### Example 5—Transmission Electron Microscopy

The single droplet-negative staining procedure was used as described previously [17] with the following modifications. 20 μl of a suspension at approximately 10⁹ bacteria/ml were absorbed for 2 min. onto form formvard carbon-coated nickel grids (400 mesh; Electron Microscopy Sciences EMS, Washington, Pa.). After 30 seconds air-drying the grids were stained for 2 minutes with 20 μl of 2% phosphotungstic acid (pH7; EMS) and examined after air-drying under a transmission electron microscope (Hitachi 7500, Japan) at 60 kvolts and high resolution.

### Example 6—Intranasal Infection and Vaccination

3-week and 8-week old female Balb/C were kept under specific pathogen-free conditions, and all experiments were carried out under the guidelines of the Institut Pasteur de Lille animal study board. Mice were intranasally infected with approximately 4×10⁶ bacteria in 20 μl PBS, and kinetics of CFU in the lungs were measured as previously described [18]. For vaccination with aPV (Tetravac; Aventis-Pasteur, France), mice were immunized intraperitoneally (i.p.) with 20% of the human dose and boosted one month later using the same dose.

### Example 7—Antibody Determination

Sera were collected, and antibody titers were estimated by enzyme-linked immunosorbent assays (ELISA) as previously described [18].

### Example 8—Cytokine Assays

Spleen cells from individual mice were tested at different time points after immunization for in vitro cytokine production in response to heat-killed B. pertussis BPSM (10⁶ cells/ml), 5.0 μg/ml PTX (purified from B. pertussis BPGR4 [19] as previously described [20] and heat-inactivated at 80° C. for 20 min), 5.0 μg filamentous hemagglutinin (FHA, purified from B. pertussis BPRA [21] as previously described [22]), 5 μg/ml concanavalin A (Sigma Chemical Co., St. Louis, Mo.) or medium alone as control. Supernatants were removed from triplicate cultures after 72 h incubation at 37° C. and 5% CO₂, and IFN-γ and IL-5 concentrations were determined by immunoassays (BD OptEIA set, Pharmingen).

### Example 9—Intranasal Infection and Vaccination: Challenge at 1, 2, 3 and 4 Weeks

An infant (3 weeks-old) mouse model [29] was used to compare the efficiency of vaccination with BPZE1 with the one of vaccination with acellular pertussis vaccine (aPv). Female Balb/C mice were intranasally infected with approximately 1×10⁶ BPZE1 strain in 20 μl PBS. For vaccination with aPv (Tetravac; Aventis-Pasteur, France), mice were immunized intraperitoneally with 20% of the human dose. One, two, three or four weeks after vaccination with BPZE1 or aPv, mice were intranasally challenged with virulent B. pertussis BPSM/betA-lacZ strain [53]. This strain is a BPSM-derivative gentamycin-resistant which allows the discrimination with BPZE1 (gentamycin-sensitive) on Bordet-Gengou agar plates containing 10 μg/ml of gentamycin and 100 μg/ml of streptomycin (BGgs). Control group corresponds to naive mice challenged with BPSM/bctA-lacZ. One week after challenge infection, lungs were aseptically removed, homogenized and plates on BGgs for CFU determination as previously described [18].

Mice were vaccinated with BPZE1 or aPv and challenged with virulent B. pertussis one, two, three or four weeks after vaccination. Lung CFUs counts were determined 3 hours or 7 days later. Results are expressed as mean (±standard error) CFUs from three to five mice per group. Levels of protection are calculated for each challenge infection as mean percentages of CFUs of each group relative of the average of CFUs in non-immunized group, 7 days after challenge infection (Tables 2 to 5).

### Example 10—Statistical Analysis

The results were analyzed using the unpaired Student's t test and the Kruskal-Wallis test followed by the Dunn's post-test (GraphPad Prism program) when appropriate. Differences were considered significant at P≤0.05.

### Results

### Construction of B. pertussis BPZE1

Three virulence factors were genetically targeted: tracheal cytotoxin (TCT), pertussis toxin (PTX) and dermonecrotic toxin (DNT).

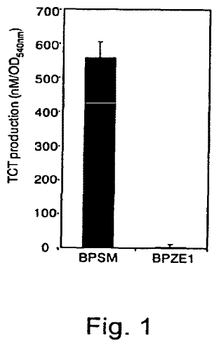

TCT is responsible for the destruction of ciliated cells in the trachea of infected hosts [24, 25] and may thus be involved in the cough syndrome. TCT is a breakdown product of peptidoglycan in the cell wall of Gram-negative bacteria, which generally internalize it into the cytosol by the AmpG transporter protein to be re-utilized during cell wall biosynthesis. B. pertussis AmpG is inefficient in the internalization of peptidoglycan breakdown products. We therefore replaced the B. pertussis ampG gene by E. coli ampG. The resulting strain expressed less than 1% residual TCT activity (FIG. 1).

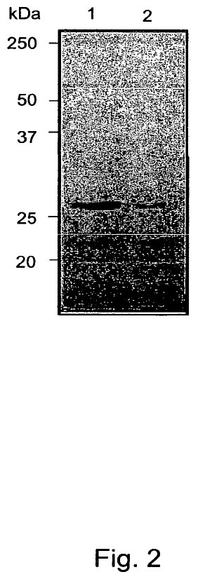

PTX is a major virulence factor responsible for the systemic effects of B. pertussis infections and is composed of an enzymatically active moiety, called S1, and a moiety responsible for binding to target cell receptors (for review, see 26). However, it is also one of the major protective antigens, which has prompted us to replace the natural ptx genes by a mutated version coding for an enzymatically inactive toxin. This was achieved by replacing Arg-9 by Lys and Glu-129 by Gly in S1, two key residues involved in substrate binding and catalysis, respectively. Allelic exchange was used to first delete the ptx operon, and then to insert the mutated version. The presence of the relevant toxin analogues in the B. pertussis culture supernatants was evaluated by immunoblot analysis (FIG. 2).

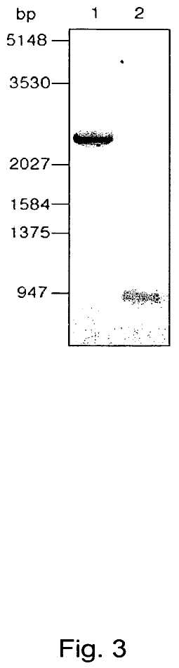

Finally, allelic exchange was used to remove the dnt gene (FIG. 3). Although the role of DNT in the virulence of B. pertussis is not certain, it has been identified as an important toxin in the closely related species Bordetella bronchiseptica and displays lethal activity upon injection of minute quantities (for review, see 26).

### In Vitro Characterization of B. pertussis BPZE1

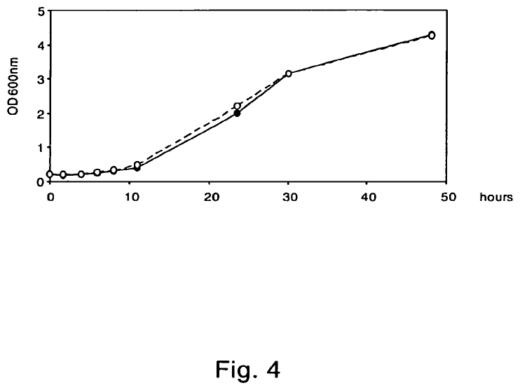

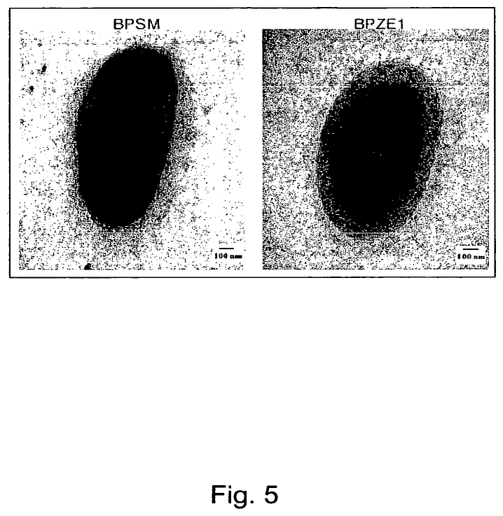

Since some of the genetic alterations in BPZE1 may potentially affect the bacterial cell wall synthesis, the size and shape, as well as the in vitro growth rate of BPZE1 was compared with those of the parental strain BPSM. The growth rate of BPZE1 did not differ from that of BPSM (FIG. 4), and no difference in bacterial shape or size was detected between BPZE1 and BPSM, as evidenced by electron microscopy analysis (FIG. 5). However, the cell wall of BPZE1 appeared to be consistently somewhat thinner than that of BPSM.

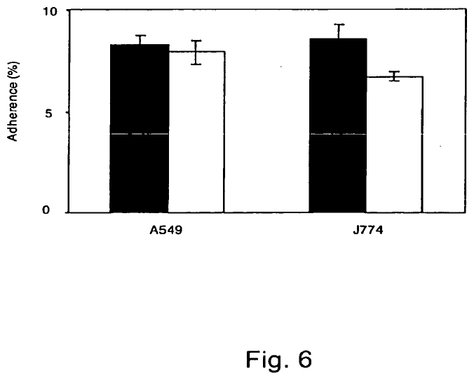

To determine whether the absence or alterations of any of the targeted toxins in BPZE1 affects adherence properties of B. pertussis, the attachment rates of BPZE1 was compared with those of BPSM, using the human pulmonary epithelial cell line A549 and the murine macrophage cell line J774, as two cellular models often used to study the adherence of B. pertussis. No significant difference in the adherence capacities to either cell line was observed between the two strains (FIG. 6).

### Attenuation of B. pertussis BPZE1

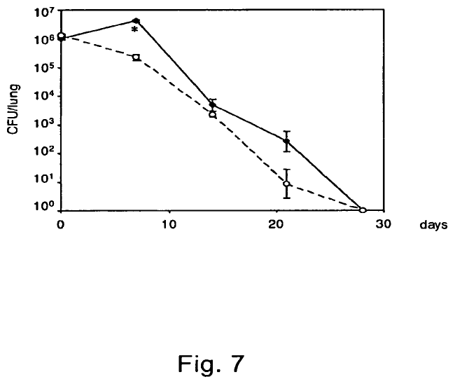

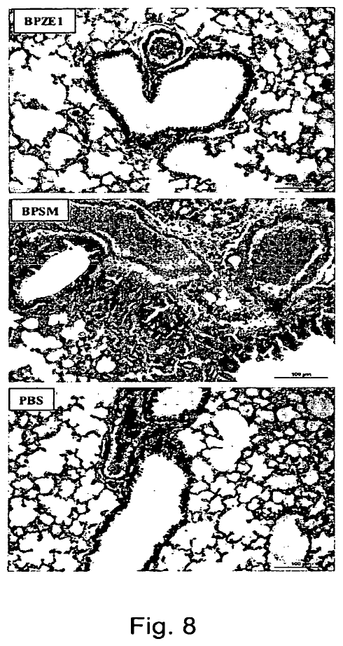

To determine whether the mutations introduced into B. pertussis BPZE1 have resulted in attenuation, yet allow the organism to colonize the respiratory tract, Balb/C mice were intranasally infected with BPZE1 or BPSM, and colonization was followed over time. BPZE1 was able to colonize and persist in the lungs of mice as long as BPSM (FIG. 7). However, the peak of multiplication seen 7 days after infection with BPSM was consistently lacking in mice infected with BPZE1. Studies done with strains mutated in individual toxin genes indicated that this is due to the mutations in the pix locus (data not shown). When the lungs were examined for histopathological changes and inflammatory infiltration, infection with BPSM was found to induce strong peri-bronchiovascular infiltrates and inflammatory cell recruitment 7 days after infection, associated with a strong hypertrophy of the bronchiolar epithelial cells (FIG. 8). In contrast, no such changes were seen in BPZE1-infected animals, and the histology of the BPZE1-infected mice was similar to that of the control mice that had received PBS instead of the bacteria. The BPSM-infection induced inflammation lasted for at least two months (data not shown). These results indicate that the mutations introduced into BPZE1 have resulted in drastic attenuation, but allow the bacteria to colonize and persist in the lungs.

### Protection Against B. pertussis Challenge After Intranasal Vaccination of Adult Mice With BPZE1

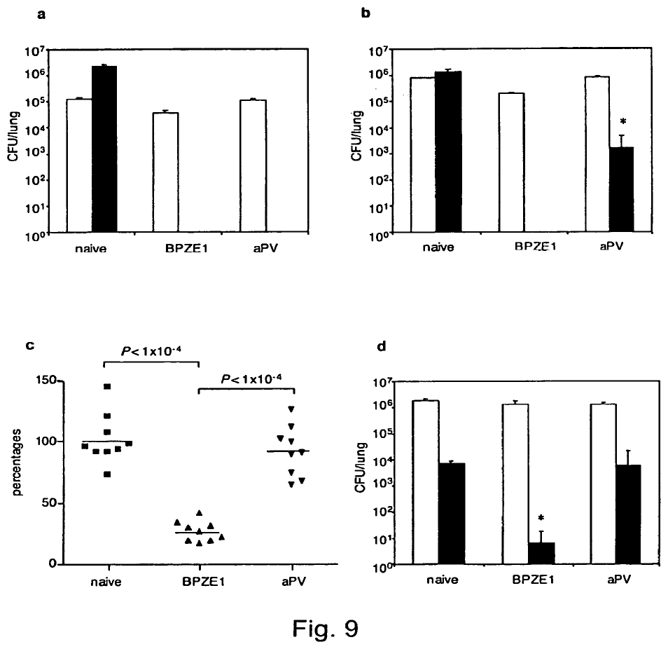

To evaluate the protection offered by BPZE1, the effect of a single intranasal administration of this strain to 8-weeks old Balb/C mice on the subsequent colonization by the wild type challenge strain BPSM was compared with that of two i. p. immunizations with ⅕ of a human dose of aPV. This aPV immunization protocol has been described as the best to correlate with pertussis vaccine efficacy in human clinical trials [27, 28]. As shown by the total clearance of bacterial colony counts in the lungs seven days after challenge infection, a single intranasal administration of BPZE1 and two i.p. immunizations with aPV provided similar levels of protection (FIG. 9a). High bacterial loads were found in the control mice that had received two injections of PBS instead of the vaccine.

### Protection Against B. pertussis Challenge After Intranasal Vaccination of Infant Mice With BPZE1

Since the principal targets of novel pertussis vaccines are young infants, that are not protected with the currently available vaccines, an infant (3 weeks-old) mouse model [29] was developed and used to compare the efficiency of vaccination with BPZE1 with that of vaccination with aPV. A single nasal administration of BPZE1 fully protected infant mice against challenge infection (FIG. 9b), as complete bacterial clearance was observed in the lungs one week after challenge. In contrast, substantial numbers of bacteria remained in the aPV-vaccinated animals one week after challenge infection. The difference in bacterial load between the BPZE1-vaccinated and the aPV-vaccinated mice was statistically significant, indicating that in the infant mouse model a single intranasal administration with BPZE1 provides better protection than two systemic administrations of aPV.

In addition, a strong reduction in the bacterial load of the challenge strain 3 hours after administration when the mice had been immunized with BPZE1 was consistently observed compared to the aPV-immunized animals (FIG. 9c), indicating that vaccination with BPZE1 reduces the susceptibility to infection by the challenge strain. This effect was seen in both 8-weeks old and in infant mice. In contrast, aPV had no effect on the bacterial counts 3 hours after infection, when compared to the control mice.

### Protection Against B. parapertussis Challenge After Intranasal Vaccination With BPZE1

There is increasing concern about B. parapertussis infection in children, especially in immunized populations [30, 31]. B. parapertussis causes a milder pertussis like syndrome, the frequency of which is probably largely underestimated. Furthermore, the incidence of B. parapertussis infections has been increasing over the last decades, possibly due to the fact that pertussis vaccines are known to have very low or no protective efficacy against B. parapertussis [32, 33]. In contrast, infection by B. pertussis has recently been reported to protect against B. parapertussis infection [34]. BPZE1 was also assessed for protection against B. parapertussis using the infant mouse model. Whereas two administrations of aPV did not provide any protection against B. parapertussis, as previously reported, a single intranasal administration of BPZE1 provided strong protection, as measured by the low numbers of B. parapertussis counts in the lungs of the vaccinated mice 1 week after challenge (FIG. 9d).

### Immune Responses Induced by BPZE1 Vaccination

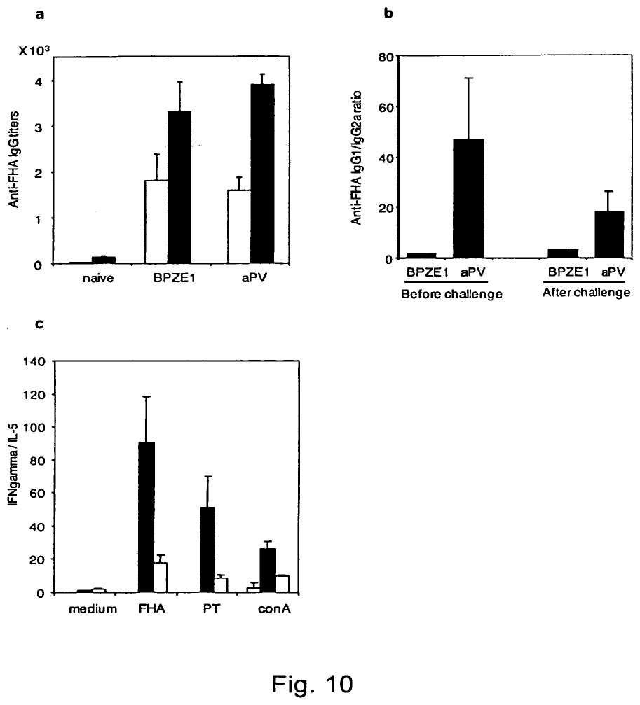

Although the mechanisms of protective immunity against B. pertussis infection are not yet completely understood, clear evidence of a role for both B cells and IFN-γ has been demonstrated in mice [28]. Vaccination with either one nasal dose of BPZE1 or two i. p. administrations of aPV induced high titers of serum IgG against FHA, a major surface antigen of B. pertussis [35], also present in aPV (FIG. 10a). Following B. pertussis challenge, positive anamnestic responses were measured in BPZE1- and in aPV-vaccinated animals, as indicated by an increase in anti-FHA IgG titers, compared to primary responses before B. pertussis infection. Examination of the anti-FHA IgG1/IgG2a ratios showed that these ratios were higher after aPV administration, characteristic of a Th2 type response, than after BPZE1 vaccination (FIG. 10b). Although the anti-FHA-IgG1/IgG2a decreased after challenge in the aPV vaccinated mice, it remained still substantially higher than in the BPZE1-vaccinated animals after B. pertussis challenge.

Analysis of B. pertussis antigen-specific cytokine patterns induced by BPZE1 or aPV vaccination confirmed that BPZE1 administration favors a stronger Th1 type response than aPV vaccination. This was revealed by the fact that the ratios of IFN-γ over IL-5 produced by splenocytes stimulated with FHA or PT, or with the polyclonal activator ConA were significantly higher in BPZE1 vaccinated mice than in aPV vaccinated mice (FIG. 10c).

### Protective Immunity of BPZE1 Over Time (From 1 Week to 4 Weeks)

As shown in Tables 1 to 5 below, whereas administration of aPv provided limited protection (reduction of 75% of bacterial load compared to non-vaccinated mice at 1 week) against B. pertussis, a single intranasal administration of BPZE1 already provided high level of protection (reduction of 97.64% of bacterial load) against a B. pertussis challenge infection performed one week after vaccination. If challenge infection occurred two weeks after vaccination, the level of protection induced by BPZE1 reached more than 99.999% compared to non-vaccinated mice and is significantly superior to the protection induced by aPv vaccine (approximately 92% compared to non-vaccinated mice). Therefore, vaccine efficacy of BPZE1 against B. pertussis challenge is already significant one week after vaccination and is increasing over the at least next two months.

TABLE 1
Kinetics of vaccines efficacy against
B. pertussis challenge in infant mice.

| | | Time | | | | |
|---|---|---|---|---|---|---|
| Time | | between | | | | |
| between | | lungs | | | | |
| vaccination | | recovery | | Log10 cfu/lungs of mice | | |
| and | | and | | | aPv- | BPZE1- |
| challenge | | challenge | | Naive | vaccinated | vaccinated |
| 1 | week | 3 | hours | 5.71 ± 0.03 | 5.8 ± 0.07 | 5.74 ± 0.01 |
| | | 7 | days | 6.71 ± 0.06 | 5.97 ± 0.20 | 4.86 ± 0.35 |
| 2 | weeks | 3 | hours | 5.77 ± 0.10 | 5.60 ± 0.02 | 5.49 ± 0.05 |
| | | 7 | days | 6.49 ± 0.08 | 5.31 ± 0.16 | 3.22 ± 0.33 |
| 3 | weeks | 3 | hours | 6.03 ± 0.11 | 5.88 ± 0.04 | 5.33 ± 0.08 |
| | | 7 | days | 6.58 ± 0.09 | 5.62 ± 0.11 | 3.14 ± 0.38 |
| 4 | weeks | 3 | hours | 6.31 ± 0.01 | 6.15 ± 0.02 | 5.83 ± 0.05 |
| | | 7 | days | 6.36 ± 0.04 | 5.21 ± 0.11 | 1.83 ± 0.46 |

TABLE 2
Level of protection of aPv-vaccinated and BPZE1-vaccinated
mice as compared to non-vaccinated mice at week 1.

| | Number of | |
|---|---|---|
| | bacteria | Mean number |
| Non vaccinated mice | in lungs | of bacteria |
| Non-vaccinated 1 | 4.7 × 10⁶ | 5.36.10⁶ |
| Non-vaccinated 2 | 3.8 × 10⁶ | |
| Non-vaccinated 3 | 8.2 × 10⁶ | |
| Non-vaccinated 4 | 4.1 × 10⁶ | |
| Non-vaccinated 5 | 6 × 10⁶ | |

| | | | |
|---|---|---|---|
| | | Mean | |
| Number of | Percentage | percentage | |
| bacteria | of remaining | of remaining | Level of |
| in lungs | bacteria ⁽¹⁾ | bacteria | protection |

aPv-vaccinated mice

| | | | | |
|---|---|---|---|---|
| aPv1 | 1.95 × 10⁶ | 36.38 | 25% | 75% |
| aPv2 | 2.9 × 10⁶ | 54.1 | | |
| aPv3 | 2.9 × 10⁵ | 5.41 | | |
| aPv4 | 3.6 × 10⁵ | 6.72 | | |
| aPv5 | 1.2 × 10⁶ | 22.39 | | |

BPZE1-vaccinated mice

| | | | | |
|---|---|---|---|---|
| BPZE1-1 | 3.2 × 10⁵ | 5.97 | 2.36% | 97.64% |
| BPZE1-2 | 2 × 10⁴ | 0.004 | | |
| BPZE1-3 | 6 × 10⁴ | 1.12 | | |

⁽¹⁾ Percentage of remaining bacteria = number of bacteria for each particular mouse/mean number of bacteria of all non-vaccinated mice

TABLE 3
Level of protection of aPv-vaccinated and BPZE1-vaccinated
mice as compared to non-vaccinated mice at week 2.

| | Number of | |
|---|---|---|
| | bacteria | Mean number |
| Non vaccinated mice | in lungs | of bacteria |
| Non-vaccinated 1 | 5 × 10⁶ | 3.34 × 10⁶ |
| Non-vaccinated 2 | 3.6 × 10⁶ | |
| Non-vaccinated 3 | 1.7 × 10⁶ | |
| Non-vaccinated 4 | 2.4 × 10⁶ | |
| Non-vaccinated 5 | 4 × 10⁶ | |

| | | | |
|---|---|---|---|
| | | Mean | |
| Number of | Percentage | percentage | |
| bacteria | of remaining | of remaining | Level of |
| in lungs | bacteria ⁽¹⁾ | bacteria | protection |

aPv-vaccinated mice

| | | | | |
|---|---|---|---|---|
| aPv1 | 9.5 × 10⁴ | 2.84 | 8.11% | 91.89% |
| aPv2 | 2.9 × 10⁵ | 8.68 | | |
| aPv3 | 1 × 10⁵ | 2.99 | | |
| aPv4 | 6.8 × 10⁵ | 20.36 | | |
| aPv5 | 1.9 × 10⁵ | 5.69 | | |

BPZE1-vaccinated mice

| | | | | |
|---|---|---|---|---|
| BPZE1-1 | 9.5 × 10³ | 2.8 × 10⁻³ | 1.03 × 10⁻³% | 99.999% |
| BPZE1-2 | 450 | 1.35 × 10⁻⁴ | | |
| BPZE1-3 | 3500 | 1.05 × 10⁻³ | | |
| BPZE1-4 | 500 | 1.5 × 10⁻⁴ | | |

⁽¹⁾ Percentage of remaining bacteria = number of bacteria for each particular mouse/mean number of bacteria of all non-vaccinated mice

TABLE 4
Level of protection of aPv-vaccinated and BPZE1-vaccinated
mice as compared to non-vaccinated mice at week 3.

| | Number of | |
|---|---|---|
| | bacteria | Mean number |
| Non vaccinated mice | in lungs | of bacteria |
| Non-vaccinated 1 | 1.8 × 10⁶ | 4.04 × 10⁶ |
| Non-vaccinated 2 | 5.75 × 10⁶ | |
| Non-vaccinated 3 | 4.7 × 10⁶ | |
| Non-vaccinated 4 | 3.2 × 10⁶ | |
| Non-vaccinated 5 | 4.75 × 10⁶ | |

| | | | |
|---|---|---|---|
| | | Mean | |
| Number of | Percentage | percentage | |
| bacteria | of remaining | of remaining | Level of |
| in lungs | bacteria ⁽¹⁾ | bacteria | protection |

aPv-vaccinated mice

| | | | | |
|---|---|---|---|---|
| aPv1 | 1.99 × 10⁵ | 4.94 | 11.26% | 88.74% |
| aPv2 | 6 × 10⁵ | 14.85 | | |
| aPv3 | 6 × 10⁵ | 14.85 | | |
| aPv4 | 4.2 × 10⁵ | 10.40 | | |

BPZE1-vaccinated mice

| | | | | |
|---|---|---|---|---|
| BPZE1-1 | 3640 | 9.01 × 10⁻⁴ | 8.65 × 10⁻⁴% | 99.999% |
| BPZE1-2 | 9720 | 2.4 × 10⁻³ | | |
| BPZE1-3 | 300 | 7.43 × 10⁻⁵ | | |
| BPZE1-4 | 340 | 8.42 × 10⁻⁵ | | |

⁽¹⁾ Percentage of remaining bacteria = number of bacteria for each particular mouse/mean number of bacteria of all non-vaccinated mice

TABLE 5
Level of protection of aPv-vaccinated and BP2E1-vaccinated
mice as compared to non-vaccinated mice at week 4.

| | Number of | |
|---|---|---|
| | bacteria | Mean number |
| Non vaccinated mice | in lungs | of bacteria |
| Non-vaccinated 1 | 2.1 × 10⁶ | 2.36 × 10⁶ |
| Non-vaccinated 2 | 2.2 × 10⁶ | |
| Non-vaccinated 3 | 3.1 × 10⁶ | |
| Non-vaccinated 4 | 2.6 × 10⁶ | |
| Non-vaccinated 5 | 1.8 × 10⁶ | |

| | | | |
|---|---|---|---|
| | | Mean | |
| Number of | Percentage | percentage | |
| bacteria | of remaining | of remaining | Level of |
| in lungs | bacteria ⁽¹⁾ | bacteria | protection |

aPv-vaccinated mice

| | | | | |
|---|---|---|---|---|
| aPv1 | 2.52 × 10⁵ | 10.68 | 7.76% | 92.24% |
| aPv2 | 3.28 × 10⁵ | 13.90 | | |
| aPv3 | 1.04 × 10⁵ | 4.41 | | |
| aPv4 | 8.4 × 10⁵ | 3.56 | | |
| aPv5 | 1.48 × 10⁵ | 6.27 | | |

BPZE1-vaccinated mice

| | | | | |
|---|---|---|---|---|
| BPZE1-1 | 190 | 8.05 × 10⁻⁵ | 7.13 × 10⁻⁵% | 99.999% |
| BPZE1-2 | 0 | 0 | | |
| BPZE1-3 | 110 | 4.66 × 10⁻⁵ | | |
| BPZE1-4 | 320 | 1.36 × 10⁻⁴ | | |
| BPZE1-5 | 220 | 9.32 × 10⁻⁵ | | |

⁽¹⁾ Percentage of remaining bacteria = number of bacteria for each particular mouse/mean number of bacteria of all non-vaccinated mice

### DISCUSSION

Pertussis is the first infectious disease whose incidence is increasing in countries with high vaccine coverage. This paradoxical situation is most likely linked to the epidemiological changes observed since the massive introduction of highly efficacious vaccines. In contrast to the pre-vaccination era, cases of adolescent and adult pertussis are now increasingly more frequent. Although generally not life-threatening in that age group, B. pertussis-infected adults are an important reservoir for infection of the very young children, too young to be protected by vaccination. Early vaccination, possibly at birth, would therefore be highly desirable, but is hampered by the immaturity of the immune system of neonates and infants. However, the fact that natural B. pertussis infection, even very early in life, is able to induce a strong Th1 response in infants [12] prompted us to develop a live attenuated B. pertussis vaccine strain to be given by the nasal route as an alternative over the currently available vaccines.

Based on experimental infections of primates, Huang et al. had already in 1962 come to the conclusion that ultimate protection against whooping cough probably best follows a live B. pertussis inoculation [36]. In veterinary medicine, attenuated Bordetella strains have been used to vaccinate against bordetellosis in dogs and piglets. A live attenuated Bordetella bronchiseptica strain has been shown to provide strong protection against kennel cough in dogs [37] after nasal administration. This protection was seen as early as 48 h after vaccination. Intranasal vaccination with live attenuated B. bronchiseptica has also been shown to protect against atrophic rhinitis in two-days old piglets [38], indicating that in a live attenuated form Bordetella vaccines can be highly active in new-born animals.

Previous attempts to genetically attenuate B. pertussis as a live vaccine candidate have met with rather limited success. Based on a strategy used for the successful attenuation of Salmonella vaccine strains [39]. Roberts et al. have deleted the aroA gene of B. pertussis [40]. The aroA mutant was indeed highly attenuated, but had also lost its capacity to colonize the respiratory tract of the intranasally vaccinated animals and induced protective immunity only after repeated administrations of high doses. We took advantage of the knowledge on the molecular mechanisms of B. pertussis virulence and developed the highly attenuated strain BPZE1. This strain contains genetic alterations leading to the absence or inactivation of three major toxins, PTX, TCT and DNT. In contrast to the aroA mutant, this strain was able to colonize the mouse respiratory tract and to provide full protection after a single intranasal administration. The protection in adult mice was indistinguishable from that induced by two administrations of ⅕ of a human dose of aPV. An important difference, however, was seen in infant mice, where a single administration of BPZE1 fully protected, whereas aPV only offered partial protection. In the context of the difficulties to induce protection in infants with the currently available vaccines early in life, these results provide hope for the development of novel vaccination strategies that may be used in the very young children, possibly at birth. In addition, BPZE1 protected against B. parapertussis, whereas aPV did not. Therefore the use of BPZE1 should also have an impact on the incidence of whooping cough caused by B. parapertussis in infants.

Although the recent replacement of first generation whole-cell vaccines by new aPV in many countries has significantly reduced the systemic adverse reactions observed with whole-cell vaccines, it has not abolished the need for repeated vaccination to achieve protection. This makes it unlikely to obtain protection in very young children (<6 months) that present the highest risk to develop severe disease. In addition, the wide-spread use of aPV has revealed new, unforeseen problems. Repeated administration of aPV may cause extensive swelling at the site of injection [41], which was not observed with whole-cell vaccines. In approximately 5% of the cases this swelling involves almost the entire limb and lasts for more than a week. Although the mechanism of this swelling has not been characterized yet, it has been proposed to be due to an Arthus hypersensitivity reaction caused by high antibody levels induced by the primary immunization [42]. However, it could also be related to the Th2 skewing of the immune response, as, compared to whole-cell vaccines, aPV administration induces more Th2-type cytokines in vaccinated children [10] and causes a delay in the Th1 development (Mascart et al., in preparation). Delayed maturation of Th1 function has been associated with a risk for atopy in genetically pre-disposed individuals [33]. The two mechanisms are not mutually exclusive. Compared to aPV, the immune response to BPZE1 administration is less biased towards the Th2 arm, and since BPZE1 is administered mucosally, no swelling reaction can occur.

The use of live attenuated bacteria as vaccines raises the issue of their biosafety. As such, they fall under the directives and guidelines for genetically modified organisms susceptible to be released into the environment. These guidelines and directives describe several objectives that have to be met, including hazard identification and environmental risk assessment [44]. Potential pathogenicity needs to be carefully considered, especially in immuno-compromised individuals, such as those infected with HIV. The natural biology of B. pertussis is particularly interesting in that regard. Although pertussis in HIV-infected subjects has been described occasionally, it is rather rare in AIDS patients [45]. In its genetically attenuated form, B. pertussis would therefore not be expected to cause major problems in HIV-infected children, especially if severe AIDS is an exclusion criterion, as it is for many vaccines. B. pertussis colonization is strictly limited to the respiratory epithelium, without extrapulmonary dissemination of the bacteria, which naturally excludes systemic bacteremia of the BPZE1 vaccine strain. If nevertheless unforeseeable safety problems occurred, the vaccine strain can easily be eliminated by the use of macrolide antibiotics, such as erythromycin, to which essentially all B. pertussis isolates are highly sensitive.

A further concern, like for any live vaccine, is the potential release of the vaccine strain in the environment and the consequences of such a release. B. pertussis is a strictly human pathogen, and there is no animal vector or reservoir. Moreover, unlike B. bronchiseptica, survival of wild-type B. pertussis in the environment is extremely limited [46]. Pertussis is only spread by coughing individuals, and there appears to be no asymptomatic carriage [47]. Coughing cannot be assessed in the mouse models used in this study. However, due to the nature of the genetic alterations in BPZE1, in particular the strong reduction of TCT and the genetic inactivation of PTX, this strain would not be expected to induce coughing. Active PTX has been shown to be required for cough induction in a coughing rat model, although the mechanism is not known [48]. If the vaccine strain were nevertheless to be transmitted to non-vaccinated individuals, this would at worst result in increased vaccine coverage. The consequences of each of these potential hazards can thus be graded as negligible and can easily and rapidly be controlled by antibiotic treatment if necessary.

Advantages of the use of BPZE1 include the relatively low production costs, making it especially attractive for developing countries, its needle-free easy and safe mode of administration and its potential to induce mucosal immunity in addition to systemic immunity. Although the role of mucosal immunity against pertussis has surprisingly not been much addressed, the fact that B. pertussis is a strictly mucosal pathogen, makes it likely that mucosal immune responses may contribute significantly to protection. None of the currently available vaccines induces any significant mucosal response.

Other advantages of the use of BPZE1 in vaccination are:
the rapid protective immune response obtained after a single intranasal dose of BPZE1, since induction of the immunity can be detected 1 week after vaccination,
an increase of the protective immunity over the at least next two months after vaccination, and
the complete protective immunity, since a level of protection of more than 99.999% is obtained 2 weeks after vaccination.

The use of live attenuated B. pertussis for mucosal vaccination offers yet another advantage. B. pertussis can be used for the presentation of heterologous antigens to the respiratory mucosa (for review see 49). The use of BPZE1 as a heterologous expression platform may thus be helpful for the generation of multivalent vaccines against a variety of respiratory pathogens. However, since intranasal delivery of BPZE1 also induces strong systemic immune responses, as shown here by both the high levels of anti-FHA antibodies and of antigen-specific IFN-γ production, it may also be used for the production of antigens to which systemic immune responses are desired.

While the invention has been described in terms of various preferred embodiments, the skilled artisan will appreciate that various modifications, substitutions, omissions and changes may be made without departing from the scope thereof. Accordingly, it is intended that the scope of the present invention be limited by the scope of the following claims, including equivalents thereof.

### REFERENCES

1. WHO (2004) The world health report 2004—changing history, Geneva, WHO.
2. Das P (2002) Whooping cough makes global comeback, Lancet ii: 322.
3. Tan T, Trindade E, Skowronski D (2005) Epidemiology of Pertussis. Pediatr Infect Dis J 24: S10-S18.
4. Centers for Disease Control and Prevention. Epidemiology and Prevention of Vaccine-Preventable Diseases. Atkinson W. Wolfe S, Hamborsky J. McIntyre L, eds. 9th ed. Washington DC: Public Health Foundation, 2006; Pertussis Chapter 14.
5. Wirsing von König C H, Halperin S, Riffelmann M, Guiso N (2002) Pertussis of adults and infants. Lancet Infect Dis 2:744-750.
6. Lewis D B, Yu C C, Meyer J, English B K, Kahn S J, et al. (1991) Cellular and molecular mechanisms for reduced interleukin-4 and interferon-γ production by neonatal T cells. J Clin Invest 87: 194-202.
7. Siegrist C A (2001) Neonatal and early life vaccinology. Vaccine. 19: 3331-3346.
8. Mills K H G (2001) Immunity to Bordetella pertussis. Microbes Infect 3: 655-677.
9. Lewis D B, Larsen A, Wilson C B (1986) Reduced interferon-γ mRNA levels in human neonates. J Exp Med 163:1018-1023.
10. Ausiello C M, Urbani F, La Sala A, Lande R, Cassone A (1997) Vaccine- and antigen-dependent type 1 and type 2 cytokine induction after primary vaccination in infants with whole-cell or acellular pertussis vaccines. Infect Immun 65: 2168-2174.
11. Wirsing von König C H, Postels-Multani S, Bock H L, Schmitt H J (1995) Pertussis in adults: frequency of transmission after household exposure. Lancet 346:1326-1329.
12. Mascart F, Verscheure V, Malfroot A, Hainaut M, Piérard D, et al. (2003) Bordetella pertussis infection in 2-months-old infants promotes Type I T cell responses. J Immunol 170:1504-1509.
13. Menozzi F D, Mutombo R, Renauld G, Gantiez C, Hannah J H, et al. (1994) Heparin-inhibitable lectin activity of the filamentous hemagglutinin adhesin of Bordetella pertussis. Infect Immun 62: 769-778.
14. Imaizumi A, Suzuki Y, Ono S, Sato H, Sato Y (1983) Effect of heptakis (2,6-O)-dimethyl)-beta-cyclodextrin on the production of pertussis toxin by Bordetella pertussis. Infect Immun 41: 1138-1143.
15. Cookson B T, Cho H-L, Herwaldt L A, Goldman W E (1989) Biological activities and chemical composition of purified tracheal cytotoxin of Bordetella pertussis. Infect Immun 57: 2223-2229.
16. Alonso S, Pethe K, Mielcarek N, Raze D, Locht C (2001) Role of ADP-ribosyltransferase activity of pertussis toxin in toxin-adhesin redundancy with filamentous hemagglutinin during Bordetella pertussis infection. Infect Immun 69: 6038-6043.
17. Collyn F, Lety M A, Nair S, Escuyer V, Ben Younes A, et al. (2002) Yersinia pseudotuberculosis harbors a type IV pilus gene cluster that contributes to pathogenicity. Infect Immun 70: 619-620.
18. Mielcarek N, Comette J, Schacht A M, Pierce R J, Locht C, et al. (1997) Intranasal priming with recombinant Bordetella pertussis for the induction of a systemic immune response against a heterologous antigen. Infect Immun 65: 544-550.
19. Locht C, Geoffroy M C, Renauld G (1992) Common accessory genes for the Bordetella pertussis filamentous hemagglutinin and fimbriae share sequence similarities with the papC and papD gene families. EMBO J. 11: 3175-3183.
20. Sekura R D, Fish F, Manclark C R, Meade B, Zhang Y L (1983) Pertussis toxin. Affinity purification of a new ADP-ribosyltransferase. J Biol Chem 258: 14647-14651.
21. Antoine R, Locht C (1990) Roles of the disulfide bond and the carboxy-terminal region of the S1 subunit in the assembly and biosynthesis of pertussis toxin. Infect Immun 58:1518-1526.
22. Menozzi F O, Gantiez C, Locht C (1991) Interaction of the Bordetella pertussis filamentous haemagglutinin with heparin. FEMS Microbiol Lett 62: 59-64.
23. Locht C, Antoine R, Jacob-Dubuisson F (2001) Bordetella pertussis, molecular pathogenesis under multiple aspects. Curr Opin Microbiol 4: 82-89.
24. Heiss L N, Flak T A, Lancaster J R, McDaniel M L, Goldman W E (1993) Nitric oxide mediates Bordetella pertussis tracheal cytotoxin damage to the respiratory epithelium. Infect Agents Dis 2: 173-177.
25. Goldman W E, Cookson B T (1988) Structure and functions of the Bordetella tracheal cytotoxin. Tokai J Exp Clin Med 13 Suppl: 187-191.
26. Locht C, Antoine R (1999) Bordetella pertussis protein toxins. In: Alouf J E, Freer J H, editors. Comprehensive sourcebook of bacterial protein toxins. Academic Press, pp. 130-146.
27. Guiso N, Capiau C, Carletti G, Poolman J, Hauser P (1999) Intranasal murine model of Bordetella pertussis infection. I. Prediction of protection in human infants by acellular vaccines. Vaccine 17: 2366-2376.
28. Mills K H, Ryan M, Ryan E, Mahon B P (1998) A murine model in which protection correlates with pertussis vaccine efficacy in children reveals complementary roles for humoral and cell-mediated immunity in protection against Bordetella pertussis. Infect Immun 66: 594-602.
29. Roduit C, Bozzotti P, Mielcarek N, Lambert P H, Del Giudice G, et al. (2002) Immunogenicity and protective efficacy of neonatal immunization against Bordetella pertussis in a murine model: Evidence for early control of Pertussis. Infect Immun 70: 3521-3528.
30. He Q, Viljanen M K, Arvilommi H, Aittanen B, Mertsola J (1998) Whooping cough caused by Bordetella pertussis and Bordetella parapertussis in an immunized population. JAMA 280: 635-637.
31. Watanabe M, Nagai M (2004) Whooping cough due to Bordetella parapertussis: an unresolved problem. Expert Rev Anti Infect Ther 2: 447-454.
32. Mastrantonio P, Stefanelli P, Giuliano M, Herrera Rojas Y, Ciofi degli Atti M, et al. (1998) Bordetella parapertussis infection in children: epidemiology, clinical symptoms, and molecular characteristics of isolates. J Clin Microbiol 36: 999-1002.
33. Liese J G, Renner C, Stojanov S, Belohradsky B H, Munich Vaccine Study Group. (2003) Clinical and epidemiological picture of B. pertussis and B. parapertussis infections after introduction of acellular pertussis vaccines. Arch Dis Child 88: 684-687.
34. Watanabe M, Nagai M (2001) Reciprocal protective immunity against Bordetella pertussis and Bordetella parapertussis in a murine model of respiratory infection. Infect Immun 69: 6981-6986.
35. Locht C, Bertin P, Menozzi F D, Renauld G (1993) The filamentous haemagglutinin, a multifaceted adhesin produced by virulent Bordetella spp. Mol Microbiol 9: 653-660.
36. Huang C C, Chen P M, Kuo J K, Chui W H, Lin S T, et al. (1962) Experimental whooping cough. N Engl J Med 266: 105-111.
37. Bey R F, Shade F J, Goodnow R A, Johnson R C (1981) Intranasal vaccination of dogs with live avirulent Bordetella bronchiseptica: correlation of serum aggutination titer and the formation of secretory IgA with protection against experimentally induced infectious tracheobronchitis. Am J Vet Res 42: 1130-1132.
38. De Jong M F (1987) Prevention of atrophic rhinitis in piglets by means of intranasal administration of a live non-AR-pathogenic Bordetella bronchiseptica vaccine. Vet Q 9:123-133.
39. Hoiseth S K, Stocker B A D (1981) Aromatic-dependent Salmonella typhimurium are non-virulent and effective as live vaccines. Nature 291: 238-239.
40. Roberts M, Maskell D, Novotny P, Dougan G (1990) Construction and characterization in vivo of Bordetella pertussis aroA mutants. Infect Immun 58: 732-739.
41. Rennels M B (2003) Extensive swelling reactions occurring after booster doses of diphtheria-tetanus-acellular pertussis vaccines. Semin Pediatr Infect Dis 14: 196-198.
42. Robbins J B, Schneerson R, Trollfors B, Sato H, Sato Y, et al. (2005) The diphtheria and pertussis components of diphtheria-tetanus toxoids-pertussis vaccine should be genetically inactivated mutant toxins. J Infect Dis 191: 81-88.
43. Holt P G, Clough J B, Holt B J, Baron-Hay M J U, Rose A H, et al. (1992) Genetic “risk” for atopy is associated with delayed postnatal maturation of T-cell competence. Clin Exp Allergy 22: 1093-1099.
44. Favre D, Viret J F (2006) Biosafety evaluation of recombinant live oral bacterial vaccines in the context of European regulation. Vaccine. May 1; 24 (18):3856-64.
45. Cohn S E, Knorr K L, Gilligan P H, Smiley M L, Weber D J (1993) Pertussis is rare in human immunodeficiency virus disease. Am Rev Respir Dis 147: 411-413.
46. Porter J F, Wardlaw A C (1993) Long-term survival of Bordetella bronchiseptica in lakewater and in buffered saline without added nutrients. FEMS Microbiol Lett 110: 33-36.
47. Linnemann C C Jr, Bass J W, Smith M H D (1968) The carrier state in pertussis. Am J Epidemiol 88: 422-427.
48. Parton R, Hall E, Wardlaw A C (1994) Responses to Bordetella pertussis mutant strains and to vaccination in the coughing rat model of pertussis. J Med Microbiol 40: 307-312.
49. Mielcarek N, Alonso S, Locht C (2001) Nasal vaccination using live bacterial vectors. Adv Drug Del Rev 51: 55-69.
50. Lyon R S, Engle J T, Goldman W E. Manuscript in preparation
51. Simon R, Priefer U, Pühler A (1983) A broad host range mobilization system for in vivo genetic engineering: transposon mutagenesis in Gram-negative bacteria. Bio/Technology 1: 784-791.
52. Stibitz S (1994) Use of conditionally counterselectable suicide vectors for allelic exchange. Methods Enzymol 235: 458-465.
53. Antoine R, Huvent I, Chemial K, Deray I, Raze D, et al. (2005) The periplasmic binding protein of tripartite tricarboxylate transporter is involved in signal transduction. J Mol Biol 351: 799-809.
54. Sato H, Ito A, Chiba J, Sato Y (1984) Monoclonal antibodies against pertussis toxin: effect on toxin activity and pertussis infections. Infect Immun 46: 422-428.
55. Sato H, Sato Y, Ito A, Ohishi I (1987) Effect of monoclonal antibody to pertussis toxin on toxin activity. Infect Immun 55: 909-915.
56. Tuomanen, E. And Weiss A. (1985) Characterization of two adhesions of Bordetella pertussis for human ciliated respiratory epithelial cells. J. Infect. Dis. 152:118-125.
57. Locht, C., Antoine, R., Veithen A. and Raze D. 2000. Pertussis Toxin: Structure-Function-Relationship. In Aktories K. Just I editors. Handbook of Experimental Pharmacology, Bacterial Protein Toxins, Springer, vol 145, pp. 167-185.
58. Horiguchi Y, Matsuda, H. Koyama H, Nakai T and Kume K. (1992) Bordetella bronchiseptica dermonecrotizing toxin suppresses in vivo antibody responses in mice. FEMS Microbiol. Lett. 69:229-234.
59. Bordet et Genysa (1909) L'endotoxine coquelucheuse; Ann. Inst. Pasteur 23: 415-419.
60. Iida & Okonogi (1971) Lieno toxicity of Bordetella pertussis in mice; J. Med. Microbiol. 4: 51-61.
61. R. Parton (1985) Effect of prednisone on the toxicity of Bordetella pertussis in mice, J. Med. Microbiol. 19: 391-400.
62. Magyar et al (1988) The pathogenesis of turbinate atrophy in pigs caused by Bordetella bronchiseptica, Vet. Microbiol. 3: 1719-1728.
63. Roop et al (1987) Virulence factors of Bordetella bronchiseptica associated with the production of infectious atropic rhinitis and pneumonia in experimentally infected neonatal swine, Infect. Immun. 55: 217-222.
64. Weiss & Goodman (1989) Lethal infection by Bordetella pertussis mutants in the infant mouse model, Infect. Immun. 57: 3757-3764.
65. Allan & Maskell (1996) The identification, cloning and mutagenesis of a genetic locus required for lipopacysaccharide biosynthesis in Bordetella pertussis, Mol. Microbiol. 19: 37-52.
66. Alonso et al (2002) Eighty kilodalton N-terminal moiety of Bordetella pertussis filamentous hemagglutinin: adherence, immunogenicity, and protective role, Infection & Immunity, 70, 4142-4147.
67. Cummings, C. A., Bootsma, H. J., Relman D. A. and Miller J. F. (2006) Species- and Strain-specific Control of a Complex, Flexible Regulon by Bordetella BvgAS. J. Bacteriol. 188:1775-1785.
68. Kashimoto T., Katahira J, Cornejo W R, Masuda M, Fukuoh A, Matsuzawa T, Ohnishi T, Horiguchi Y. (1999) Identification of functional domains of Bordetella dermonecrotizing toxin. Infect. Immun. 67 (8) 3727-32.

What is claimed is:

1. A method of vaccinating a mammalian subject to induce a more Th1-biased immune response against Bordetella pertussis than that induced using an acellular pertussis vaccine (aPV), the method comprising the step of administering a dose of a vaccine comprising a sufficient number of a live attenuated Bordetella pertussis strain to the respiratory tract of the subject to cause colonization of the subjects lungs with the live attenuated Bordetella pertussis strain and cause a more Th1-biased immune response against Bordetella pertussis compared to the immune response against Bordetella pertussis that would have been induced in the subject had the subject been vaccinated with an aPV instead of the live attenuated Bordetella pertussis strain-based vaccine.

2. The method of claim 1, wherein the colonization of the subject's lungs with the live attenuated Bordetella pertussis strain causes the subject's splenocytes to secrete a higher ratio of gamma interferon (IFNγ) to interleukin 5 (IL-5) when activated with a Bordetella antigen selected from pertussis toxin and filamentous hemagglutinin (FHA) compared to ratio of IFNγ to IL-5 secreted by the subject's splenocytes had the subject been vaccinated with an aPV instead of the live attenuated Bordetella pertussis strain-based vaccine.

3. The method of claim 1, wherein the colonization of the subject's lungs with the live attenuated Bordetella pertussis strain induces an increase in the titer of anti-FHA IgG in the subject's serum comparable to the increase in the titer of anti-FHA IgG in the subject's serum that would have been induced in the subject had the subject been vaccinated with an aPV instead of the live attenuated Bordetella pertussis strain-based vaccine.

4. The method of claim 3, wherein the serum anti-FHA IgG1/IgG2a ratio induced by the colonization of the subject's lungs with the live attenuated Bordetella pertussis strain is lower than the serum anti-FHA IgG1/IgG2a ratio that would have been induced in the subject had the subject been vaccinated with an aPV instead of the live attenuated Bordetella pertussis strain-based vaccine.

5. The method of claim 1, wherein the colonization of the subject's lungs with the live attenuated Bordetella pertussis strain induces an immune response in the subject which reduces the amount of inflammatory cell infiltration into the lungs of the subject caused by infection of the subject with a pathogenic strain of Bordetella pertussis after the subject has been colonized with the live attenuated Bordetella pertussis strain compared to the amount of inflammatory cell infiltration into the lungs of the subject caused by infection of the subject with a pathogenic strain of Bordetella pertussis had the subject's lungs not been colonized with the live attenuated Bordetella pertussis strain before being infected with the pathogenic strain of Bordetella pertussis.

6. The method of claim 5, wherein administering to the subject a single dose of the vaccine comprising the live attenuated Bordetella pertussis strain one week before the subject is infected by the pathogenic strain of Bordetella pertussis reduces the number of pathogenic Bordetella pertussis bacteria in the lungs of the subject seven days after being infected with the pathogenic strain of Bordetella pertussis compared to the number of pathogenic Bordetella pertussis bacteria in the lungs of the subject seven days after being infected with the pathogenic strain of Bordetella pertussis had the subject been vaccinated with a single dose of aPV instead of the single dose of the vaccine comprising the live attenuated Bordetella pertussis strain.

7. The method of claim 5, wherein administering to the subject a single dose of the vaccine comprising the live attenuated Bordetella pertussis strain one week before the subject is infected by the pathogenic strain of Bordetella pertussis reduces the number of pathogenic Bordetella pertussis bacteria in the lungs of the subject twenty-eight days after being infected with the pathogenic strain of Bordetella pertussis compared to the number of pathogenic Bordetella pertussis bacteria in the lungs of the subject twenty-eight days after being infected with the pathogenic strain of Bordetella pertussis had the subject been vaccinated with a single dose of aPV instead of the single dose of the vaccine comprising the live attenuated Bordetella pertussis strain.

8. The method of claim 7, wherein the number of pathogenic Bordetella pertussis bacteria in the lungs of the subject twenty-eight days after being infected with the pathogenic strain of Bordetella pertussis is at least one thousand-fold less than the number of pathogenic Bordetella pertussis bacteria in the lungs of the subject twenty eight days after being infected with the pathogenic strain of Bordetella pertussis had the subject been vaccinated with a single dose of aPV instead of the single dose of the vaccine comprising the live attenuated Bordetella pertussis strain.

9. The method of claim 1, wherein live attenuated Bordetella pertussis strain produces an immunogenic but enzymatically inactive pertussis toxin (PTX) but not an enzymatically active PTX.

10. The method of claim 9, wherein the live attenuated Bordetella pertussis strain comprises a heterologous ampG gene replacing the Bordetella ampG gene.

11. The method of claim 9, wherein the live attenuated Bordetella pertussis strain comprises a deleted or mutated dermonecrotic toxin gene.

12. The method of claim 10, wherein the live attenuated Bordetella pertussis strain comprises a deleted or mutated dermonecrotic toxin gene.

13. A method of inducing a more Th1-biased immune response against Bordetella pertussis in a mammalian subject than that induced using an acellular pertussis vaccine (aPV), the method comprising the step of colonizing the respiratory tract of the subject with a sufficient amount of a live attenuated Bordetella pertussis-based means for eliciting an anti-Bordetella pertussis immune response, wherein such colonization causes the subject to develop a more Th1-biased immune response against Bordetella pertussis compared to the immune response against Bordetella pertussis that would have been induced in the subject had the subject been vaccinated with an aPV instead of the live attenuated Bordetella pertussis-based vaccine means.
